(19) United States
(12) Patent Application Publication
(10) Pub. No.: US 2024/0368680 A1
(43) Pub. Date: Nov. 7, 2024

(54) COMPOSITIONS AND METHODS FOR DECROSSLINKING BIOLOGICAL SAMPLES

(71) Applicant: Promega Corporation, Madison, WI (US)

(72) Inventors: Hui Wang, Madison, WI (US); Kevin Mayer, Madison, WI (US); Douglas Horejsh, Madison, WI (US); Marjeta Urh, Madison, WI (US); Wenhui Zhou, Madison, WI (US); Michael M. Rosenblatt, Madison, WI (US)

(21) Appl. No.: 18/654,939

(22) Filed: May 3, 2024

Related U.S. Application Data
(60) Provisional application No. 63/500,180, filed on May 4, 2023.

(51) Int. Cl. C12Q 1/6851; C12Q 1/6806; G01N 1/30

(52) U.S. Cl. CPC C12Q 1/6851; C12Q 1/6806; G01N 1/30

(57) ABSTRACT

Disclosed herein are compositions and methods for decrosslinking formaldehyde cross-linked biological samples such as formalin-fixed, paraffin-embedded (FFPE) tissue samples.

### CROSS-REFERENCE TO RELATED APPLICATIONS

This application claims priority to and the benefit of U.S. Provisional Patent Application No. 63/500,180, filed on May 4, 2023, which is incorporated herein by reference in its entirety.

### FIELD

Provided herein are compositions and methods for decrosslinking formaldehyde cross-linked biological samples such as formalin-fixed, paraffin-embedded (FFPE) tissue samples.

### BACKGROUND

Formalin Fixed Paraffin Embedded (FFPE) tissue specimens are precious clinical samples that contain a great deal of patient information. When such samples are prepared, formalin acts as a preservative by creating extensive molecular crosslinks. Before nucleic acids, proteins, and other biomolecules can be extracted and used in downstream assays, the crosslinks must be reversed, which requires significant preprocessing time. Typically, after deparaffinization and lysis, tissues are decrosslinked for long periods (hours to days) at high temperatures (60-90° C.). While this decrosslinking step is necessary, it also can contribute to extraction failure and poor sample quality, which poses a significant challenge for clinical and research applications.

### SUMMARY

In one aspect, disclosed herein is a method of decrosslinking a formaldehyde cross-linked biological sample, comprising contacting the sample with an effective amount of a compound of formula (I):

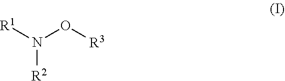

or a salt thereof,
wherein:
   (i) R¹ is selected from H, C₁-C₆ alkyl, and carboxy-C₁-C₄-alkyl:
   R² is selected from H, C₁-C₆ alkyl, hydroxy-C₁-C₄-alkyl, carboxy-C₁-C₄-alkyl, aryl, heteroaryl, aryl-C₁-C₄-alkyl, and heteroaryl-C₁-C₄-alkyl:
   R³ is —X—R⁴, wherein X is selected from —C(O)— and —SO₂—, and R⁴ is selected from C₁-C₆-alkyl, aryl, and heteroaryl:
   wherein each aryl and heteroaryl is independently unsubstituted or substituted with 1 or 2 substituents independently selected from halo, hydroxy, C₁-C₄ alkyl, C₁-C₄ alkoxy, amino, amido, carboxy, and ester; and
   wherein each alkyl is unsubstituted or substituted with 1 or 2 substituents independently selected from halo, hydroxy, alkoxy, amino, amido, carboxy, ester, optionally substituted C₃-C₆ cycloalkyl, and optionally substituted 3- to 6-membered heterocyclyl:
or
   (ii) R¹ is selected from H, C₁-C₆ alkyl, and carboxy-C₁-C₄-alkyl:
   R² is selected from C₁-C₆ alkyl, aryl-C₁-C₄-alkyl, heteroaryl-C₁-C₄-alkyl, and —Y—R⁵, wherein Y is selected from a bond, —C(O)—, and —SO₂—, and R⁵ is selected from C₁-C₆ alkyl, aryl, and heteroaryl; and
   R³ is H:
   wherein each aryl and heteroaryl is independently unsubstituted or substituted with 1 or 2 substituents independently selected from halo, hydroxy, C₁-C₄ alkyl, C₁-C₄ alkoxy, amino, amido, carboxy, and ester; and
   wherein each alkyl is unsubstituted or substituted with 1 or 2 substituents independently selected from halo, hydroxy, alkoxy, amino, amido, carboxy, ester, optionally substituted C₃-C₆ cycloalkyl, and optionally substituted 3- to 6-membered heterocyclyl:
or
   (iii) R¹ and R², together with the nitrogen atom to which they are attached, are taken together to form an optionally substituted 4- to 8-membered ring; and R³ is H:
or
   (iv) R¹ is H; and
   R² and R³, together with the atoms to which they are attached, are taken together to form an optionally substituted 4- to 8-membered ring.

In some embodiments: R¹ is H: R² is selected from C₁-C₆ alkyl, —CH₂-aryl, and —CH₂— heteroaryl; and R³ is —X—R⁴, wherein X is —C(O)— and R⁴ is selected from C₁-C₆-alkyl, aryl, and heteroaryl; wherein each alkyl is independently unsubstituted or substituted with 1 substituent selected from hydroxy and carboxy; and wherein each aryl and heteroaryl is independently unsubstituted or substituted with 1 or 2 substituents independently selected from halo, hydroxy, methoxy, amino, and carboxy.

In some embodiments: R¹ is H; R² is C₁-C₆ alkyl that is unsubstituted or substituted with 1 substituent selected from hydroxy and carboxy; and R³ is —X—R⁴, wherein X is —C(O)— and R⁴ is phenyl that is unsubstituted or substituted with 1 or 2 substituents independently selected from methoxy, hydroxy, and halo.

In some embodiments: R¹ is H; R² is selected from aryl-C₁-C₄-alkyl and heteroaryl-C₁-C₄-alkyl, each of which is independently unsubstituted or substituted with 1 or 2 substituents independently selected from halo, hydroxy, methoxy, amino, and carboxy; and R³ is H.

In some embodiments: R¹ and R², together with the nitrogen atom to which they are attached, are taken together form a saturated 4- to 6-membered ring that is unsubstituted or substituted with 1 or 2 substituents independently selected from hydroxy, hydroxy-C₁-C₄ alkyl, C₁-C₄-alkoxy, carboxy, carboxy-C₁-C₄ alkyl, and oxo; and R³ is H.

In some embodiments: R¹ is H; and R² and R³, together with the atoms to which they are attached, are taken together to form a saturated 4- to 7-membered ring that is unsubstituted or substituted with 1 or 2 substituents independently selected from hydroxy, hydroxy-C₁-C₄ alkyl, C₁-C₄-alkoxy, carboxy, carboxy-C₁-C₄ alkyl, and oxo.

In some embodiments, the compound of formula (I) is selected from:

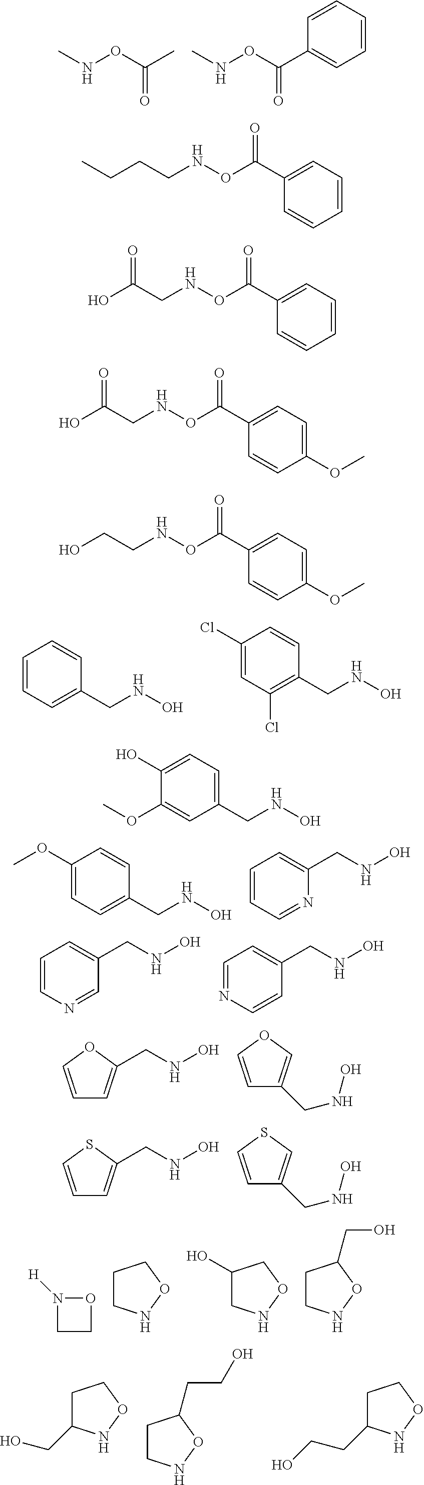

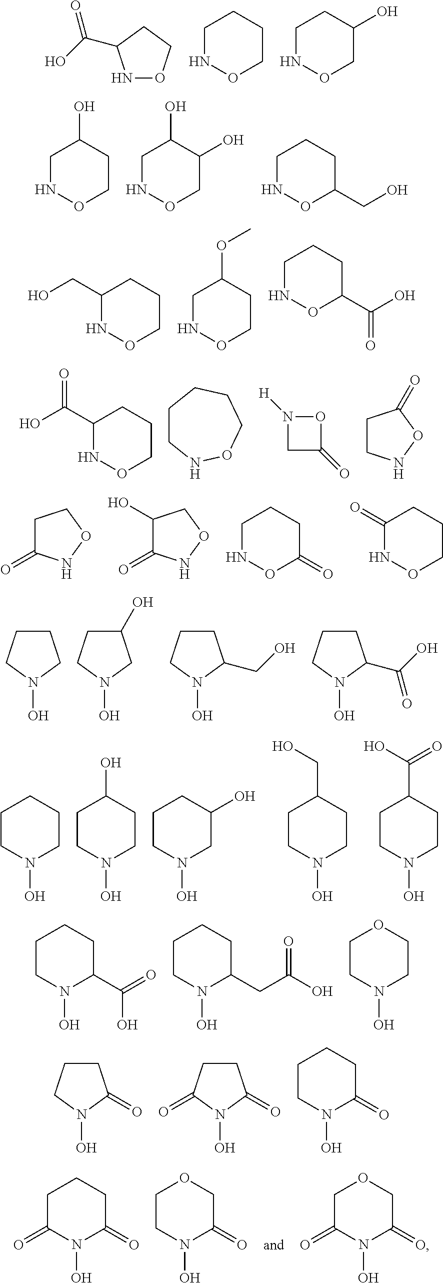

and salts thereof.

In some embodiments, the compound of formula (I) is selected from:

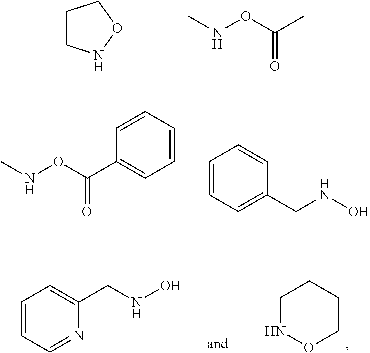

and salts thereof.

In some embodiments, the compound of formula (I) is in the form of a salt. In some embodiments, the compound of formula (I) is in the form of a hydrochloric acid salt.

In some embodiments, the method comprises contacting the sample with effective amounts of at least two different compounds of formula (I), or salts thereof. In some embodiments, the at least two different compounds of formula (I), or salts thereof, are added to the sample simultaneously. In some embodiments, the at least two different compounds of formula (I), or salts thereof, are added to the sample sequentially.

In some embodiments, the sample is a formalin-fixed paraffin embedded tissue sample. In some embodiments, the method further comprises a step of deparaffinizing the sample prior to contacting the sample with the compound of formula (I), or the salt thereof. In some embodiments, the method further comprises a step of contacting the sample with a protease prior to contacting the sample with the compound of formula (I), or the salt thereof.

In some embodiments, the protease is proteinase K.

In some embodiments, the contacting step is conducted for about 5 minutes to about 120 minutes. In some embodiments, the contacting step is conducted for about 20 minutes to about 40 minutes. In some embodiments, the contacting step is conducted at a temperature of about 20° C. to about 100° C. In some embodiments, the contacting step is conducted at a temperature of about 50° C. to about 85° C.

In some embodiments, the method comprises contacting the sample with a solution of the compound of formula (I), or the salt thereof, and the solution further comprises a buffer. In some embodiments, solution comprises a buffer selected from tris (hydroxymethyl) aminomethane (Tris), 2-(N-morpholino) ethanesulfonic acid (MES), 4-(2-hydroxyethyl)-1-piperazineethanesulfonic acid (HEPES), phosphate-buffered saline, glycine, and citrate.

In some embodiments, the method comprises contacting the sample with a solution of the compound of formula (I), or the salt thereof, and the resulting solution has a pH of about 4.0 to about 8.5 upon contacting the sample with the solution of the compound of formula (I). In some embodiments, the method comprises comprising contacting the sample with a solution of the compound of formula (I), or the salt thereof, and the resulting solution has a pH of about 4.5 to about 6.5 upon contacting the sample with the solution of the compound of formula (I).

In some embodiments, the method comprises contacting the sample with a solution of the compound of formula (I), or the salt thereof, wherein the solution comprises the compound of formula (I) at a concentration of about 1 mM to about 100 mM. In some embodiments, the method comprises contacting the sample with a solution of the compound of formula (I), or the salt thereof, wherein the solution comprises the compound of formula (I) at a concentration of about 5 mM to about 50 mM.

In some embodiments, the method further comprises extracting one or more components from the sample after the contacting step. In some embodiments, the one or more components are selected from nucleic acids and proteins. In some embodiments, the one or more components are nucleic acids, and the method further comprises a step of detecting and/or amplifying one or more nucleic acids. In some embodiments, the one or more components are proteins, and the method further comprises a step of detecting one or more proteins. In some embodiments, the detecting step comprises a colorimetric assay, fluorescence spectroscopy, UV-vis spectrophotometry, electrophoresis, an immunoassay, or mass spectrometry.

In some embodiments, the method further comprises a step selected from dye binding, absorbance, and enzymatic digestion.

In another aspect, disclosed herein is a composition comprising:
a formaldehyde cross-linked biological sample; and
a compound of formula (I):

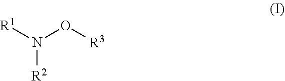

or a salt thereof,
wherein:
   (i) R¹ is selected from H, C₁-C₆ alkyl, and carboxy-C₁-C₄-alkyl:
   R² is selected from H, C₁-C₆ alkyl, hydroxy-C₁-C₄-alkyl, carboxy-C₁-C₄-alkyl, aryl, heteroaryl, aryl-C₁-C₄-alkyl, and heteroaryl-C₁-C₄-alkyl:
   R³ is —X—R⁴, wherein X is selected from —C(O)— and —SO₂—, and R⁴ is selected from C₁-C₆-alkyl, aryl, and heteroaryl:
   wherein each aryl and heteroaryl is independently unsubstituted or substituted with 1 or 2 substituents independently selected from halo, hydroxy, C₁-C₄ alkyl, C₁-C₄ alkoxy, amino, amido, carboxy, and ester; and
   wherein each alkyl is unsubstituted or substituted with 1 or 2 substituents independently selected from halo, hydroxy, alkoxy, amino, amido, carboxy, ester, optionally substituted C₃-C₆ cycloalkyl, and optionally substituted 3- to 6-membered heterocyclyl:
or
   (ii) R¹ is selected from H, C₁-C₆ alkyl, and carboxy-C₁-C₄-alkyl:
   R² is selected from C₁-C₆ alkyl, aryl-C₁-C₄-alkyl, heteroaryl-C₁-C₄-alkyl, and —Y—R⁵, wherein Y is selected from a bond, —C(O)—, and —SO₂—, and R⁵ is selected from C₁-C₆ alkyl, aryl, and heteroaryl; and
   R³ is H:
   wherein each aryl and heteroaryl is independently unsubstituted or substituted with 1 or 2 substituents independently selected from halo, hydroxy, C₁-C₄ alkyl, C₁-C₄ alkoxy, amino, amido, carboxy, and ester; and
   wherein each alkyl is unsubstituted or substituted with 1 or 2 substituents independently selected from halo, hydroxy, alkoxy, amino, amido, carboxy, ester, optionally substituted C₃-C₆ cycloalkyl, and optionally substituted 3- to 6-membered heterocyclyl:
or
   (iii) R¹ and R², together with the nitrogen atom to which they are attached, are taken together to form an optionally substituted 4- to 8-membered ring; and R³ is H:
or
   (iv) R¹ is H; and
   R² and R³, together with the atoms to which they are attached, are taken together to form an optionally substituted 4- to 8-membered ring.

In some embodiments: R¹ is H: R² is selected from C₁-C₆ alkyl, —CH₂-aryl, and —CH₂— heteroaryl; and R³ is —X—R⁴, wherein X is —C(O)— and R⁴ is selected from C₁-C₆-alkyl, aryl, and heteroaryl; wherein each alkyl is independently unsubstituted or substituted with 1 substituent selected from hydroxy and carboxy; and wherein each aryl and heteroaryl is independently unsubstituted or substituted with 1 or 2 substituents independently selected from halo, hydroxy, methoxy, amino, and carboxy.

In some embodiments: R¹ is H; R² is C₁-C₆ alkyl that is unsubstituted or substituted with 1 substituent selected from hydroxy and carboxy; and R³ is —X—R⁴, wherein X is —C(O)— and R⁴ is phenyl that is unsubstituted or substituted with 1 or 2 substituents independently selected from methoxy, hydroxy, and halo.

In some embodiments: R¹ is H: R² is selected from aryl-C₁-C₄-alkyl and heteroaryl-C₁-C₄-alkyl, each of which is independently unsubstituted or substituted with 1 or 2 substituents independently selected from halo, hydroxy, methoxy, amino, and carboxy; and R³ is H.

In some embodiments: R¹ and R², together with the nitrogen atom to which they are attached, are taken together form a saturated 4- to 6-membered ring that is unsubstituted or substituted with 1 or 2 substituents independently selected from hydroxy, hydroxy-C₁-C₄ alkyl, C₁-C₄-alkoxy, carboxy, carboxy-C₁-C₄ alkyl, and oxo; and R³ is H.

In some embodiments: R¹ is H; and R² and R³, together with the atoms to which they are attached, are taken together to form a saturated 4- to 7-membered ring that is unsubstituted or substituted with 1 or 2 substituents independently selected from hydroxy, hydroxy-C₁-C₄ alkyl, C₁-C₄-alkoxy, carboxy, carboxy-C₁-C₄ alkyl, and oxo.

In some embodiments, the compound of formula (I) is selected from:

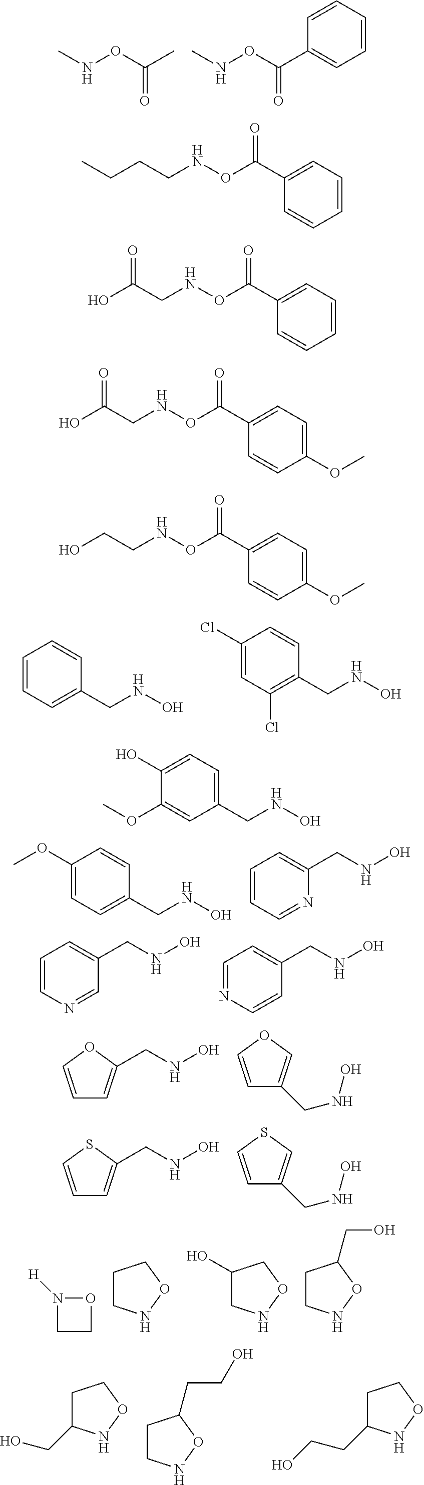

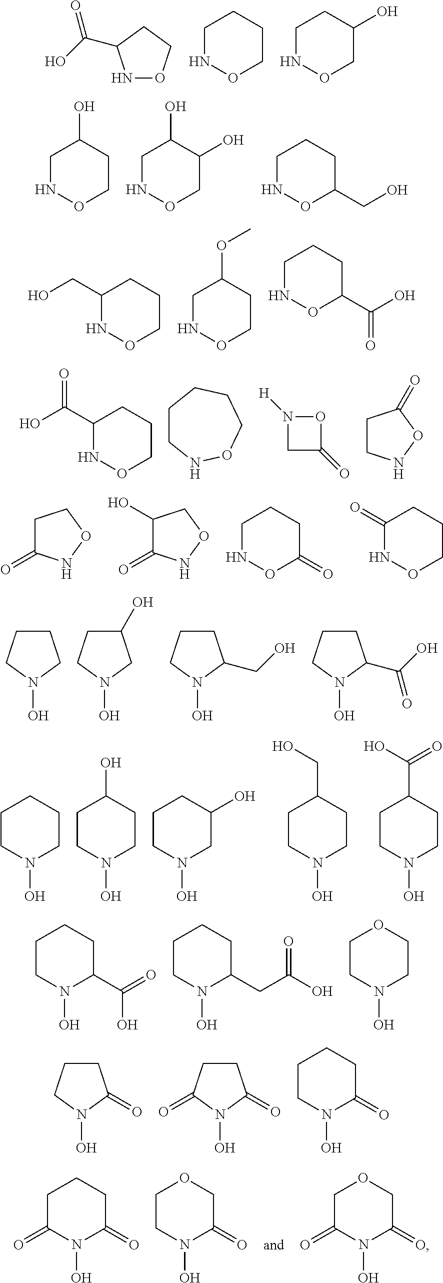

and salts thereof.

In some embodiments, the compound of formula (I) is selected from:

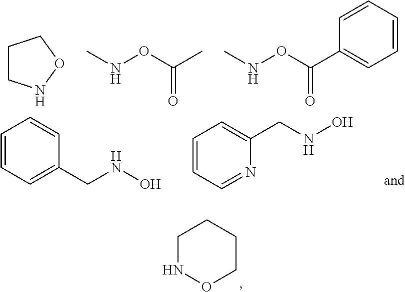

and salts thereof.

In some embodiments, the compound of formula (I) is in the form of a salt. In some embodiments, the compound of formula (I) is in the form of a hydrochloric acid salt. In some embodiments, the composition further comprises a buffer. In some embodiments, the buffer is selected from tris (hydroxymethyl) aminomethane (Tris), 2-(N-morpholino) ethanesulfonic acid (MES), 4-(2-hydroxyethyl)-1-piperazineethanesulfonic acid (HEPES), phosphate-buffered saline, glycine, and citrate

In some embodiments, the composition comprises at least two different compounds of formula (I), or salts thereof.

In some embodiments, the sample is a formalin-fixed paraffin embedded tissue sample.

In another aspect, disclosed herein is a kit comprising:

(A) a compound of formula (I):

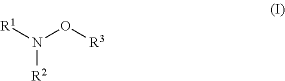

or a salt thereof,
wherein:
   (i) R¹ is selected from H, C₁-C₆ alkyl, and carboxy-C₁-C₄-alkyl;
   R² is selected from H, C₁-C₆ alkyl, hydroxy-C₁-C₄-alkyl, carboxy-C₁-C₄-alkyl, aryl, heteroaryl, aryl-C₁-C₄-alkyl, and heteroaryl-C₁-C₄-alkyl:
   R³ is —X—R⁴, wherein X is selected from —C(O)— and —SO₂—, and R⁴ is selected from C₁-C₆-alkyl, aryl, and heteroaryl:
   wherein each aryl and heteroaryl is independently unsubstituted or substituted with 1 or 2 substituents independently selected from halo, hydroxy, C₁-C₄ alkyl, C₁-C₄ alkoxy, amino, amido, carboxy, and ester; and wherein each alkyl is unsubstituted or substituted with 1 or 2 substituents independently selected from halo, hydroxy, alkoxy, amino, amido, carboxy, ester, optionally substituted C₃-C₆ cycloalkyl, and optionally substituted 3- to 6-membered heterocyclyl:
or
   (ii) R¹ is selected from H, C₁-C₆ alkyl, and carboxy-C₁-C₄-alkyl:
   R² is selected from C₁-C₆ alkyl, aryl-C₁-C₄-alkyl, heteroaryl-C₁-C₄-alkyl, and —Y—R⁵, wherein Y is selected from a bond, —C(O)—, and —SO₂—, and R⁵ is selected from C₁-C₆ alkyl, aryl, and heteroaryl; and R³ is H:
   wherein each aryl and heteroaryl is independently unsubstituted or substituted with 1 or 2 substituents independently selected from halo, hydroxy, C₁-C₄ alkyl, C₁-C₄ alkoxy, amino, amido, carboxy, and ester; and
   wherein each alkyl is unsubstituted or substituted with 1 or 2 substituents independently selected from halo, hydroxy, alkoxy, amino, amido, carboxy, ester, optionally substituted C₃-C₆ cycloalkyl, and optionally substituted 3- to 6-membered heterocyclyl:
or
   (iii) R¹ and R², together with the nitrogen atom to which they are attached, are taken together to form an optionally substituted 4- to 8-membered ring; and R³ is H;
or
   (iv) R¹ is H; and
   R² and R³, together with the atoms to which they are attached, are taken together to form an optionally substituted 4- to 8-membered ring; and
(B) instructions for decrosslinking a formaldehyde cross-linked biological sample by contacting the sample with the compound of formula (I), or the salt thereof.

In some embodiments: R¹ is H; R² is selected from C₁-C₆ alkyl, —CH₂-aryl, and —CH₂— heteroaryl; and R³ is —X—R⁴, wherein X is —C(O)— and R⁴ is selected from C₁-C₆-alkyl, aryl, and heteroaryl; wherein each alkyl is independently unsubstituted or substituted with 1 substituent selected from hydroxy and carboxy; and wherein each aryl and heteroaryl is independently unsubstituted or substituted with 1 or 2 substituents independently selected from halo, hydroxy, methoxy, amino, and carboxy.

In some embodiments: R¹ is H; R² is C₁-C₆ alkyl that is unsubstituted or substituted with 1 substituent selected from hydroxy and carboxy; and R³ is —X—R⁴, wherein X is —C(O)— and R⁴ is phenyl that is unsubstituted or substituted with 1 or 2 substituents independently selected from methoxy, hydroxy, and halo.

In some embodiments: R¹ is H; R² is selected from aryl-C₁-C₄-alkyl and heteroaryl-C₁-C₄-alkyl, each of which is independently unsubstituted or substituted with 1 or 2 substituents independently selected from halo, hydroxy, methoxy, amino, and carboxy; and R³ is H.

In some embodiments: R¹ and R², together with the nitrogen atom to which they are attached, are taken together form a saturated 4- to 6-membered ring that is unsubstituted or substituted with 1 or 2 substituents independently selected from hydroxy, hydroxy-C₁-C₄ alkyl, C₁-C₄-alkoxy, carboxy, carboxy-C₁-C₄ alkyl, and oxo; and R³ is H.

In some embodiments: R¹ is H; and R² and R³, together with the atoms to which they are attached, are taken together to form a saturated 4- to 7-membered ring that is unsubstituted or substituted with 1 or 2 substituents independently selected from hydroxy, hydroxy-C₁-C₄ alkyl, C₁-C₄-alkoxy, carboxy, carboxy-C₁-C₄ alkyl, and oxo.

In some embodiments, the compound of formula (I) is selected from:

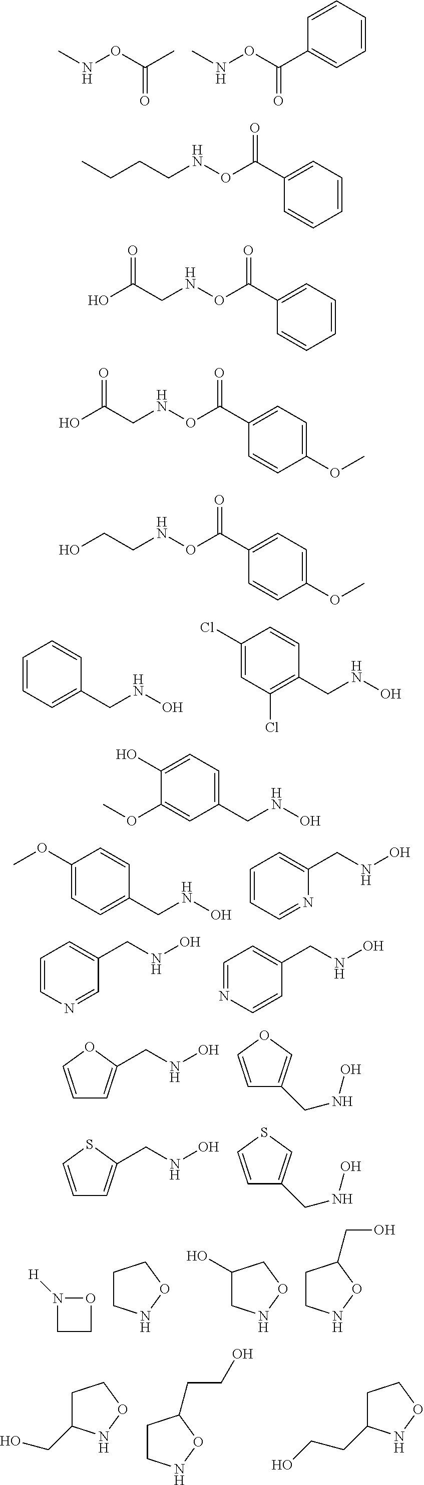

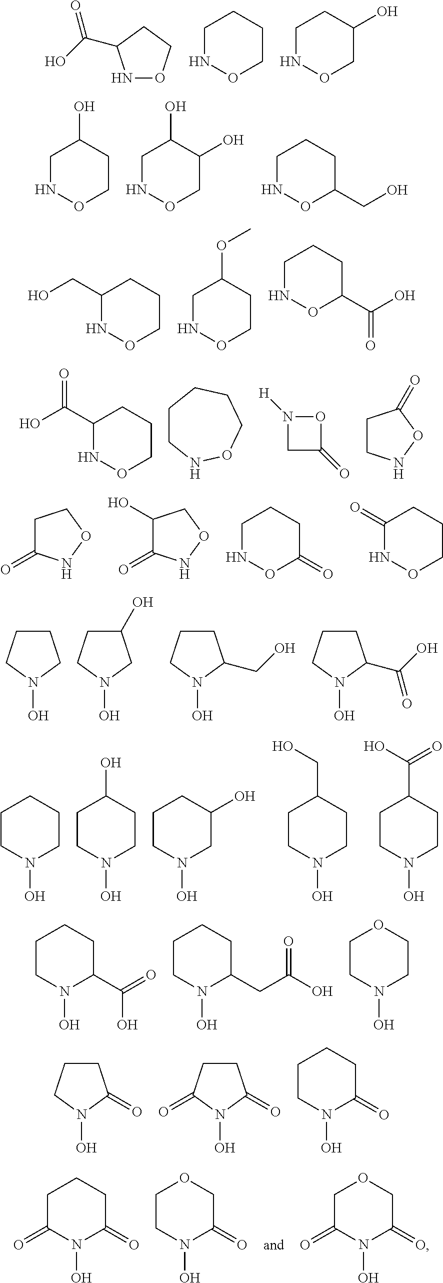

and salts thereof.

In some embodiments, the compound of formula (I) is selected from:

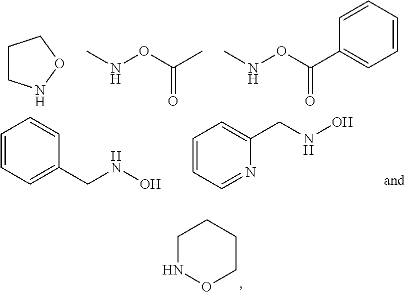

and salts thereof.

In some embodiments, the compound of formula (I) is in the form of a salt. In some embodiments, the compound of formula (I) is in the form of a hydrochloric acid salt.

In some embodiments, the composition comprises at least two different compounds of formula (I), or salts thereof.

In some embodiments, the formaldehyde cross-linked biological sample is a formalin-fixed paraffin embedded tissue sample.

In another aspect, disclosed herein is a method of decrosslinking a formaldehyde cross-linked biological sample, comprising contacting the sample with an effective amount of at least two different compounds of formula (I):

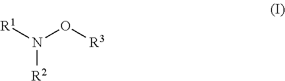

or a salt thereof,
wherein:
   (i) R¹ is selected from H, C₁-C₆ alkyl, and carboxy-C₁-C₄-alkyl;
   R² is selected from H, C₁-C₆ alkyl, hydroxy-C₁-C₄-alkyl, carboxy-C₁-C₄-alkyl, aryl, heteroaryl, aryl-C₁-C₄-alkyl, and heteroaryl-C₁-C₄-alkyl;
   R³ is —X—R⁴, wherein X is selected from —C(O)— and —SO₂—, and R⁴ is selected from C₁-C₆-alkyl, aryl, and heteroaryl;
   wherein each aryl and heteroaryl is independently unsubstituted or substituted with 1 or 2 substituents independently selected from halo, hydroxy, C₁-C₄ alkyl, C₁-C₄ alkoxy, amino, amido, carboxy, and ester; and
   wherein each alkyl is unsubstituted or substituted with 1 or 2 substituents independently selected from halo, hydroxy, alkoxy, amino, amido, carboxy, ester, optionally substituted C₃-C₆ cycloalkyl, and optionally substituted 3- to 6-membered heterocyclyl:
or
   (ii) R¹ is selected from H, C₁-C₆ alkyl, and carboxy-C₁-C₄-alkyl:
   R² is selected from C₁-C₆ alkyl, aryl-C₁-C₄-alkyl, heteroaryl-C₁-C₄-alkyl, and —Y—R⁵, wherein Y is selected from a bond, —C(O)—, and —SO₂—, and R⁵ is selected from C₁-C₆ alkyl, aryl, and heteroaryl; and
   R³ is H:
   wherein each aryl and heteroaryl is independently unsubstituted or substituted with 1 or 2 substituents independently selected from halo, hydroxy, C₁-C₄ alkyl, C₁-C₄ alkoxy, amino, amido, carboxy, and ester; and
   wherein each alkyl is unsubstituted or substituted with 1 or 2 substituents independently selected from halo, hydroxy, alkoxy, amino, amido, carboxy, ester, optionally substituted C₃-C₆ cycloalkyl, and optionally substituted 3- to 6-membered heterocyclyl:
or
   (iii) R¹ and R², together with the nitrogen atom to which they are attached, are taken together to form an optionally substituted 4- to 8-membered ring; and R³ is H:
or
   (iv) R¹ is H; and
   R² and R³, together with the atoms to which they are attached, are taken together to form an optionally substituted 4- to 8-membered ring.

In another aspect, disclosed herein is a composition comprising:
a formaldehyde cross-linked biological sample; and
at least two different compounds of formula (I):

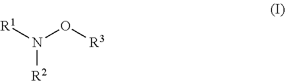

or a salt thereof,
wherein:
   (i) R¹ is selected from H, C₁-C₆ alkyl, and carboxy-C₁-C₄-alkyl:
   R² is selected from H, C₁-C₆ alkyl, hydroxy-C₁-C₄-alkyl, carboxy-C₁-C₄-alkyl, aryl, heteroaryl, aryl-C₁-C₄-alkyl, and heteroaryl-C₁-C₄-alkyl:
   R³ is —X—R⁴, wherein X is selected from —C(O)— and —SO₂—, and R⁴ is selected from C₁-C₆-alkyl, aryl, and heteroaryl:
   wherein each aryl and heteroaryl is independently unsubstituted or substituted with 1 or 2 substituents independently selected from halo, hydroxy, C₁-C₄ alkyl, C₁-C₄ alkoxy, amino, amido, carboxy, and ester; and
   wherein each alkyl is unsubstituted or substituted with 1 or 2 substituents independently selected from halo, hydroxy, alkoxy, amino, amido, carboxy, ester, optionally substituted C₃-C₆ cycloalkyl, and optionally substituted 3- to 6-membered heterocyclyl:
or
   (ii) R¹ is selected from H, C₁-C₆ alkyl, and carboxy-C₁-C₄-alkyl:
   R² is selected from C₁-C₆ alkyl, aryl-C₁-C₄-alkyl, heteroaryl-C₁-C₄-alkyl, and —Y—R⁵, wherein Y is selected from a bond, —C(O)—, and —SO₂—, and R⁵ is selected from C₁-C₆ alkyl, aryl, and heteroaryl; and
   R³ is H:
   wherein each aryl and heteroaryl is independently unsubstituted or substituted with 1 or 2 substituents independently selected from halo, hydroxy, C₁-C₄ alkyl, C₁-C₄ alkoxy, amino, amido, carboxy, and ester; and
   wherein each alkyl is unsubstituted or substituted with 1 or 2 substituents independently selected from halo, hydroxy, alkoxy, amino, amido, carboxy, ester, optionally substituted C₃-C₆ cycloalkyl, and optionally substituted 3- to 6-membered heterocyclyl:
or
   (iii) R¹ and R², together with the nitrogen atom to which they are attached, are taken together to form an optionally substituted 4- to 8-membered ring; and R³ is H:
or
   (iv) R¹ is H; and
   R² and R³, together with the atoms to which they are attached, are taken together to form an optionally substituted 4- to 8-membered ring.

In another aspect, disclosed herein is a kit comprising:
(A) at least two different compounds of formula (I):

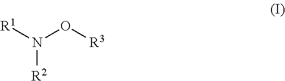

or a salt thereof,
wherein:
   (i) R¹ is selected from H, C₁-C₆ alkyl, and carboxy-C₁-C₄-alkyl;
   R² is selected from H, C₁-C₆ alkyl, hydroxy-C₁-C₄-alkyl, carboxy-C₁-C₄-alkyl, aryl, heteroaryl, aryl-C₁-C₄-alkyl, and heteroaryl-C₁-C₄-alkyl:
   R³ is —X—R⁴, wherein X is selected from —C(O)— and —SO₂—, and R⁴ is selected from C₁-C₆-alkyl, aryl, and heteroaryl:
   wherein each aryl and heteroaryl is independently unsubstituted or substituted with 1 or 2 substituents independently selected from halo, hydroxy, C₁-C₄ alkyl, C₁-C₄ alkoxy, amino, amido, carboxy, and ester; and
   wherein each alkyl is unsubstituted or substituted with 1 or 2 substituents independently selected from halo, hydroxy, alkoxy, amino, amido, carboxy, ester, optionally substituted C₃-C₆ cycloalkyl, and optionally substituted 3- to 6-membered heterocyclyl:
or
   (ii) R¹ is selected from H, C₁-C₆ alkyl, and carboxy-C₁-C₄-alkyl:
   R² is selected from C₁-C₆ alkyl, aryl-C₁-C₄-alkyl, heteroaryl-C₁-C₄-alkyl, and —Y—R⁵, wherein Y is selected from a bond, —C(O)—, and —SO₂—, and R⁵ is selected from C₁-C₆ alkyl, aryl, and heteroaryl; and
   R³ is H:
   wherein each aryl and heteroaryl is independently unsubstituted or substituted with 1 or 2 substituents independently selected from halo, hydroxy, C₁-C₄ alkyl, C₁-C₄ alkoxy, amino, amido, carboxy, and ester; and
   wherein each alkyl is unsubstituted or substituted with 1 or 2 substituents independently selected from halo, hydroxy, alkoxy, amino, amido, carboxy, ester, optionally substituted C₃-C₆ cycloalkyl, and optionally substituted 3- to 6-membered heterocyclyl:
or
   (iii) R¹ and R², together with the nitrogen atom to which they are attached, are taken together to form an optionally substituted 4- to 8-membered ring; and R³ is H;
or
   (iv) R¹ is H; and
   R² and R³, together with the atoms to which they are attached, are taken together to form an optionally substituted 4- to 8-membered ring; and
(B) instructions for decrosslinking a formaldehyde cross-linked biological sample by contacting the sample with the compound of formula (I), or the salt thereof.

### BRIEF DESCRIPTION OF THE DRAWINGS

FIG. 1 shows exemplary cross-links formed when formalin is used to fix tissues, including protein-protein crosslinks and protein-DNA crosslinks.

FIG. 2 shows exemplary workflows used to test decrosslinking catalyst compounds, along with positive and negative controls.

FIG. 3 shows quantity and quality of DNA yields purified from healthy FFPE colon tissue using decrosslinking catalyst compounds of the present disclosure along with positive and negative controls as determined by the ProNex® DNA QC Assay (75 bp, 150 bp, and 300 bp qPCR amplicons).

FIGS. 4A-4C shows quantity and quality of DNA sequencing libraries made with DNA purified in FIG. 3 using Illumina's AmpliSeq for Cancer HotSpot Panel v2. FIG. 4A shows the molecular weight of the libraries as determined by electrophoresis with Agilent TapeStation. FIG. 4B shows quantification of electrophoresed libraries. FIG. 4C shows the quantity of the libraries as determined by qPCR with ProNex® NGS Library Quantification Kit.

FIGS. 5A-5B shows concordance of single-nucleotide variant counts with DNA purified from FIG. 3, as determined by sequencing with an Illumina MiSeq. FIG. 5A shows total variant allele counts with frequencies above 1%. FIG. 5B shows variant allele frequencies between DNA purified with rapid decrosslinking catalysts, positive control, and sequencing control gDNA.

FIG. 6 shows amplifiable DNA yields from FFPE rapidly decrosslinked with catalyst compounds at 80° C. for 10, 20, and 30 minutes.

FIGS. 7A-7C show determination of optimal pH for rapid decrosslinking reactions with catalyst compounds.

FIG. 8 shows amplifiable DNA yields from FFPE rapidly decrosslinked with 20 mM, 10 mM, and 5 mM of catalyst HCl salt compounds across a range of pH lysis buffers.

FIGS. 9A-9C show purified DNA yields from a rapid decrosslinking reaction with Cpd 1. DNA was purified from thirteen human FFPE tissues and assessed using an amplification assay (FIG. 9A), a dsDNA dye-binding assay (FIG. 9B), and UV-Vis absorbance (FIG. 9C).

FIG. 10 shows a workflow for the extraction of protein from FFPE tissue and analytical techniques used.

FIG. 11A-11B shows soluble and insoluble FFPE protein yields as a function of decrosslinking incubation time at 90° C. FIG. 11A shows protein yields as determined by SDS-PAGE silver staining. FIG. 11B shows soluble protein yields as determined by a BCA assay.

FIG. 12 shows soluble protein yields from rapidly decrosslinked FFPE lysates as determined by SDS-PAGE silver staining.

FIGS. 13A-13E show soluble protein yields from FFPE lysates that were rapidly decrosslinked with catalysts across a range of pHs, as determined by SDS-PAGE silver staining.

FIGS. 14A-14B show soluble protein yields from FFPE lysates that were rapidly decrosslinked with a range of catalyst concentrations while maintaining a constant pH, as determined by SDS-PAGE silver staining.

FIGS. 15A-15C show soluble protein yields from FFPE lysates that were rapidly decrosslinked with three different incubation temperatures (65° C., 80° C., and 95° C.) and at three separate time points ranging from 10 minutes to 8 hours, as determined by SDS-PAGE silver staining.

FIG. 16 shows Histone H3 protein from FFPE lysates that were rapidly decrosslinked, as determined by western blotting.

FIGS. 17A-17C show volcano plots of rapidly decrosslinked FFPE proteins relative to a low pH control, as determined by LC-MS/MS.

FIG. 18 shows abundance of select human proteins from FIGS. 17A-17C that were enriched by the rapid decrosslinked extractions.

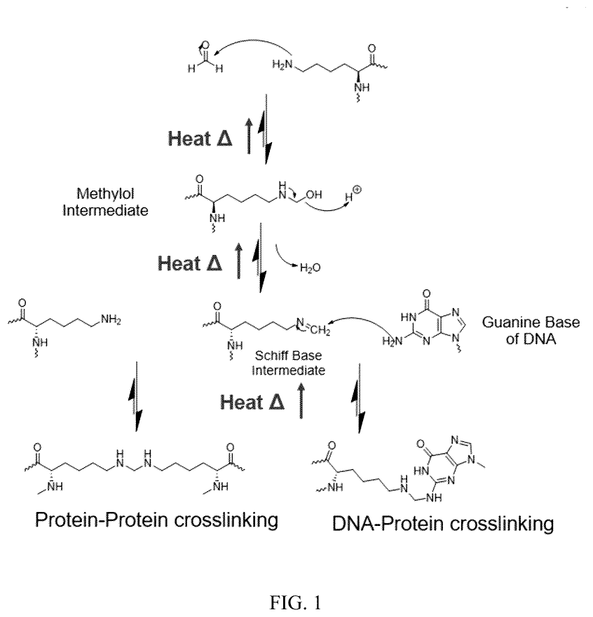

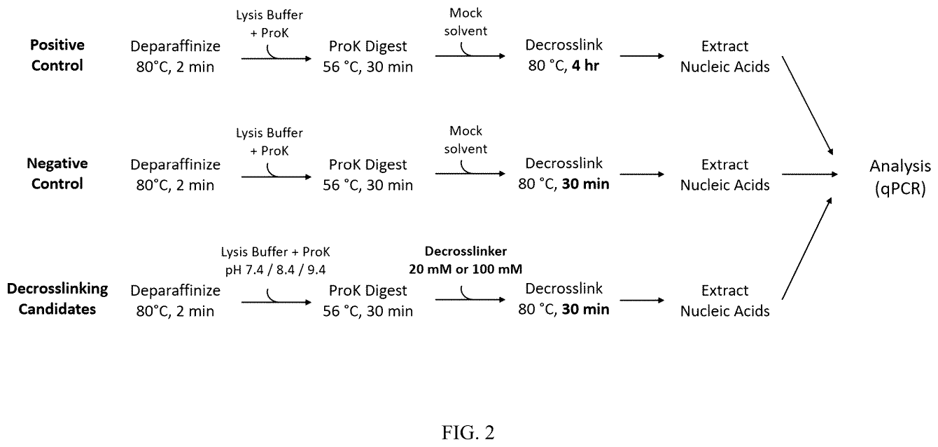

### DETAILED DESCRIPTION

Provided herein are compositions, methods, and kits for decrosslinking formaldehyde cross-linked biological samples, such as FFPE tissue samples. In some embodiments, the samples can be decrosslinked in significantly shorter times than those currently used. For example, in some embodiments, FFPE samples can be efficiently decrosslinked in only 30 minutes, without sacrificing sample quality.

### Definitions

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics, and protein and nucleic acid chemistry and hybridization described herein are those that are well known and commonly used in the art. The meaning and scope of the terms should be clear: in the event, however of any latent ambiguity, definitions provided herein take precedent over any dictionary or extrinsic definition. Further, unless otherwise required by context, singular terms shall include pluralities and plural terms shall include the singular.

As used herein and in the appended claims, the singular forms “a,” “an,” and “the” include plural reference unless the context clearly dictates otherwise.

As used herein, the term “and/or” includes any and all combinations of listed items, including any of the listed items individually. For example, “A, B, and/or C” encompasses A, B, C, AB, AC, BC, and ABC, each of which is to be considered separately described by the statement “A, B, and/or C.”

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Definitions of specific functional groups and chemical terms are described in more detail below. For purposes of this disclosure, the chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Sorrell, Organic Chemistry, 2^{nd} edition, University Science Books, Sausalito, 2006; Smith, March's Advanced Organic Chemistry:

Reactions, Mechanism, and Structure, 7th Edition, John Wiley & Sons, Inc., New York, 2013: Larock, Comprehensive Organic Transformations, 3rd Edition, John Wiley & Sons, Inc., New York, 2018; and Carruthers, Some Modern Methods of Organic Synthesis, 3rd Edition, Cambridge University Press, Cambridge, 1987; the entire contents of each of which are incorporated herein by reference.

As used herein, the term “alkyl” refers to a radical of a straight or branched saturated hydrocarbon chain. The alkyl chain can include, e.g., from 1 to 24 carbon atoms (C₁-C₂₄ alkyl), 1 to 16 carbon atoms (C₁-C₁₆ alkyl), 1 to 14 carbon atoms (C₁-C₁₄ alkyl), 1 to 12 carbon atoms (C₁-C₁₂ alkyl), 1 to 10 carbon atoms (C₁-C₁₀ alkyl), 1 to 8 carbon atoms (C₁-C₈ alkyl), 1 to 6 carbon atoms (C₁-C₆ alkyl), 1 to 4 carbon atoms (C₁-C₄ alkyl), 1 to 3 carbon atoms (C₁-C₃ alkyl), or 1 to 2 carbon atoms (C₁-C₂ alkyl). Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, n-undecyl, and n-dodecyl.

As used herein, the term “alkenyl” refers to a radical of a straight or branched hydrocarbon chain containing at least one carbon-carbon double bond and no triple bonds. The double bond(s) may be located at any position(s) with the hydrocarbon chain. The alkenyl chain can include, e.g., from 2 to 24 carbon atoms (C₂-C₂₄ alkenyl), 2 to 16 carbon atoms (C₂-C₁₆ alkenyl), 2 to 14 carbon atoms (C₂-C₁₄ alkenyl), 2 to 12 carbon atoms (C₂-C₁₂ alkenyl), 2 to 10 carbon atoms (C₂-C₁₀ alkenyl), 2 to 8 carbon atoms (C₂-C₈ alkenyl), 2 to 6 carbon atoms (C₂-C₆ alkenyl), 2 to 4 carbon atoms (C₂-C₄ alkenyl), 2 to 3 carbon atoms (C₂-C₃ alkenyl), or 2 carbon atoms (C₂ alkenyl). Representative examples of alkenyl include, but are not limited to, ethenyl, 1-propenyl, 2-propenyl, 1-butenyl, 2-butenyl, butadienyl, 2-methyl-2-propenyl, 3-butenyl, pentenyl, pentadienyl, hexenyl, heptenyl, octenyl, octatrienyl, and the like.

As used herein, the term “alkynyl” means a radical of a straight or branched hydrocarbon chain containing at least one carbon-carbon triple bond. The alkynyl chain can include, e.g., from 2 to 24 carbon atoms (C₂-C₂₄ alkynyl), 2 to 16 carbon atoms (C₂-C₁₆ alkynyl), 2 to 14 carbon atoms (C₂-C₁₄ alkynyl), 2 to 12 carbon atoms (C₂-C₁₂ alkynyl), 2 to 10 carbon atoms (C₂-C₁₀ alkynyl), 2 to 8 carbon atoms (C₂-C₈ alkynyl), 2 to 6 carbon atoms (C₂-C₆ alkynyl), 2 to 4 carbon atoms (C₂-C₄ alkynyl), 2 to 3 carbon atoms (C₂-C₃ alkynyl), or 2 carbon atoms (C₂ alkynyl). The triple bond(s) may be located at any position(s) with the hydrocarbon chain. Representative examples of alkynyl include, but are not limited to, ethynyl, 1-propynyl, 2-propynyl, 1-butynyl, 2-butynyl, and the like.

As used herein, the term “alkoxy” refers to an alkyl group, as defined herein, appended to the parent molecular moiety through an oxygen atom. Representative examples of alkoxy include, but are not limited to, methoxy, ethoxy, propoxy, 2-propoxy, butoxy, and tert-butoxy.

As used herein, the term “amino” refers to a group —NRₓR_{y}, wherein Rₓ and R_{y} are selected from hydrogen and alkyl (e.g., C₁-C₄ alkyl). A group-NH (alkyl) may be referred to herein as “alkylamino” and a group-N(alkyl) 2 may be referred to herein as “dialkylamino.”

As used herein, the term “aryl” refers to a radical of a monocyclic, bicyclic, or tricyclic 4n+2 aromatic ring system (e.g., having 6, 10, or 14 π electrons shared in a cyclic array) having 6-14 ring carbon atoms and zero heteroatoms (“C₆-C₁₄ aryl”). In some embodiments, an aryl group has six ring carbon atoms (“C₆ aryl”, i.e., phenyl). In some embodiments, an aryl group has ten ring carbon atoms (“C₁₀ aryl”, e.g., naphthyl such as 1-naphthyl and 2-naphthyl). In some embodiments, an aryl group has fourteen ring carbon atoms (“C₁₄ aryl”, e.g., anthracenyl and phenanthrenyl).

As used herein, the term “carboxy” refers to a —COOH group.

As used herein, the term “carboxyalkyl” refers to an alkyl group, as defined herein, in which at least one hydrogen atom is replaced with a carboxy group. Representative examples of carboxyalkyl include, but are not limited to, carboxymethyl, 2-carboxyethyl, and 3-carboxypropyl.

As used herein, the term “cycloalkyl” refers to a radical of a saturated carbocyclic ring system containing three to ten carbon atoms and zero heteroatoms. The cycloalkyl may be monocyclic, bicyclic, bridged, fused, or spirocyclic. Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, bicyclo[2.2.1]heptanyl, bicyclo[3.2.1]octanyl, and bicyclo [5.2.0]nonanyl.

As used herein, the term “ester” refers to a group-COOR, wherein R is an alkyl group as defined herein (e.g., a C₁-C₆, C₁-C₄, or C₁-C₃ alkyl group).

As used herein, the term “halogen” or “halo” refers to F, Cl, Br, or I.

As used herein, the term “haloalkyl” refers to an alkyl group, as defined herein, in which at least one hydrogen atom (e.g., one, two, three, four, five, six, seven or eight hydrogen atoms) is replaced with a halogen. In some embodiments, each hydrogen atom of the alkyl group is replaced with a halogen (“perhaloalkyl”). Representative examples of haloalkyl include, but are not limited to, fluoromethyl, difluoromethyl, trifluoromethyl, 2-fluoroethyl, 2,2,2-trifluoroethyl, and 3,3,3-trifluoropropyl.

As used herein, the term “heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms, wherein each heteroatom is independently selected from nitrogen, oxygen, and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). Exemplary 5-membered heteroaryl groups containing one heteroatom include, without limitation, pyrrolyl, furanyl, and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5, membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

As used herein, the term “heterocyclyl” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Exemplary 3-membered heterocyclyl groups containing one heteroatom include, without limitation, azirdinyl, oxiranyl, and thiorenyl. Exemplary 4-membered heterocyclyl groups containing one heteroatom include, without limitation, azetidinyl, oxetanyl, and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl, and pyrrolyl-2.5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl (e.g., 2.2.6.6-tetramethylpiperidinyl), tetrahydropyranyl, dihydropyridinyl, pyridinonyl (e.g., 1-methylpyridin-2-onyl), and thianyl. Exemplary 6-membered heterocyclyl groups containing two heteroatoms include, without limitation, piperazinyl, morpholinyl, pyridazinonyl (2-methylpyridazin-3-onyl), pyrimidinonyl (e.g., 1-methylpyrimidin-2-onyl, 3-methylpyrimidin-4-onyl), dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a Co aryl ring (also referred to herein as a 5.6-bicyclic heterocyclyl ring) include, without limitation, indolinyl, isoindolinyl, dihydrobenzofuranyl, dihydrobenzothienyl, benzoxazolinonyl, and the like. Exemplary 5-membered heterocyclyl groups fused to a heterocyclyl ring (also referred to herein as a 5,5-bicyclic heterocyclyl ring) include, without limitation, octahydropyrrolopyrrolyl (e.g., octahydropyrrolo[3,4-c]pyrrolyl), and the like. Exemplary 6-membered heterocyclyl groups fused to a heterocyclyl ring (also referred to as a 4,6-membered heterocyclyl ring) include, without limitation, diazaspirononanyl (e.g., 2,7-diazaspiro[3.5]nonanyl). Exemplary 6-membered heterocyclyl groups fused to an aryl ring (also referred to herein as a 6,6-bicyclic heterocyclyl ring) include, without limitation, tetrahydroquinolinyl, tetrahydroisoquinolinyl, and the like. Exemplary 6-membered heterocyclyl groups fused to a cycloalkyl ring (also referred to herein as a 6,7-bicyclic heterocyclyl ring) include, without limitation, azabicyclooctanyl (e.g., (1,5)-8-azabicyclo[3.2.1]octanyl). Exemplary 6-membered heterocyclyl groups fused to a cycloalkyl ring (also referred to herein as a 6,8-bicyclic heterocyclyl ring) include, without limitation, azabicyclononanyl (e.g., 9-azabicyclo[3.3. 1]nonanyl).

As used herein, the term “hydroxy” or “hydroxyl” refers to an —OH group.

As used herein, the term “hydroxyalkyl” refers to an alkyl group, as defined herein, in which at least one hydrogen atom is replaced with a hydroxy group. Representative examples of hydroxyalkyl include, but are not limited to, hydroxymethyl, 2-hydroxyethyl, and 3-hydroxypropyl.

As used herein, the term “oxo” refers to a group ═O.

When a group or moiety can be substituted, the term “substituted” indicates that one or more (e.g., 1, 2, 3, 4, 5, or 6; in some embodiments 1, 2, or 3; and in other embodiments 1 or 2) hydrogens on the group indicated in the expression using “substituted” can be replaced with a selection of recited indicated groups or with a suitable substituent group known to those of skill in the art (e.g., one or more of the groups recited below), provided that the designated atom's normal valence is not exceeded. Substituent groups include, but are not limited to, alkyl, alkenyl, alkynyl, alkoxy, acyl, amino, amido, amidino, aryl, azido, carbamoyl, carboxy, carboxyalkyl, cyano, cycloalkyl, cycloalkenyl, ester, guanidino, halo, haloalkyl, haloalkoxy, heteroalkyl, heteroaryl, heterocyclyl, hydroxy, hydroxyalkyl, hydrazino, imino, oxo, nitro, phosphate, phosphonate, sulfonic acid, thiol, thione, or combinations thereof.

As used herein, in chemical structures the indication:

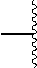

represents a point of attachment of one moiety to another moiety (e.g., a substituent group to the rest of the compound).

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

### Compounds and Compositions

Disclosed herein are compositions, methods, systems, and kits for decrosslinking formaldehyde cross-linked biological samples, such as FFPE samples. The compositions, methods, systems, and kits, described in further detail below, comprise a compound of formula (I):

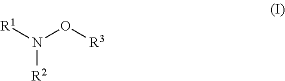

or a salt thereof,
wherein:
   (i) R¹ is selected from H, C₁-C₆ alkyl, and carboxy-C₁-C₄-alkyl:
   R² is selected from H, C₁-C₆ alkyl, hydroxy-C₁-C₄-alkyl, carboxy-C₁-C₄-alkyl, aryl, heteroaryl, aryl-C₁-C₄-alkyl, and heteroaryl-C₁-C₄-alkyl;
   R³ is —X—R⁴, wherein X is selected from —C(O)— and —SO₂—, and R⁴ is selected from C₁-C₆-alkyl, aryl, and heteroaryl:
   wherein each aryl and heteroaryl is independently unsubstituted or substituted with 1 or 2 substituents independently selected from halo, hydroxy, C₁-C₄ alkyl, C₁-C₄ alkoxy, amino, amido, carboxy, and ester; and
   wherein each alkyl is unsubstituted or substituted with 1 or 2 substituents independently selected from halo, hydroxy, alkoxy, amino, amido, carboxy, ester, optionally substituted C₃-C₆ cycloalkyl, and optionally substituted 3- to 6-membered heterocyclyl:
or
   (ii) R¹ is selected from H, C₁-C₆ alkyl, and carboxy-C₁-C₄-alkyl:
   R² is selected from C₁-C₆ alkyl, aryl-C₁-C₄-alkyl, heteroaryl-C₁-C₄-alkyl, and —Y—R⁵, wherein Y is selected from a bond, —C(O)—, and —SO₂—, and R⁵ is selected from C₁-C₆ alkyl, aryl, and heteroaryl; and
   R³ is H:
   wherein each aryl and heteroaryl is independently unsubstituted or substituted with 1 or 2 substituents independently selected from halo, hydroxy, C₁-C₄ alkyl, C₁-C₄ alkoxy, amino, amido, carboxy, and ester; and
   wherein each alkyl is unsubstituted or substituted with 1 or 2 substituents independently selected from halo, hydroxy, alkoxy, amino, amido, carboxy, ester, optionally substituted C₃-C₆ cycloalkyl, and optionally substituted 3- to 6-membered heterocyclyl:
or
   (iii) R¹ and R², together with the nitrogen atom to which they are attached, are taken together to form an optionally substituted 4- to 8-membered ring; and R³ is H:
or
   (iv) R¹ is H; and
   R² and R³, together with the atoms to which they are attached, are taken together to form an optionally substituted 4- to 8-membered ring.

In some embodiments, in the compound of formula (I) or the salt thereof:
R¹ is selected from H, C₁-C₆ alkyl, and carboxy-C₁-C₄-alkyl:
R² is selected from H, C₁-C₆ alkyl, hydroxy-C₁-C₄-alkyl, carboxy-C₁-C₄-alkyl, aryl, heteroaryl, aryl-C₁-C₄-alkyl, and heteroaryl-C₁-C₄-alkyl:
R³ is —X—R⁴, wherein X is selected from —C(O)— and —SO₂—, and R⁴ is selected from C₁-C₆-alkyl, aryl, and heteroaryl:
wherein each aryl and heteroaryl is independently unsubstituted or substituted with 1 or 2 substituents independently selected from halo, hydroxy, C₁-C₄ alkyl, C₁-C₄ alkoxy, amino, amido, carboxy, and ester; and
wherein each alkyl is unsubstituted or substituted with 1 or 2 substituents independently selected from halo, hydroxy, alkoxy, amino, amido, carboxy, ester, optionally substituted C₃-C₆ cycloalkyl, and optionally substituted 3- to 6-membered heterocyclyl.

In some embodiments, R¹ is H. In some embodiments, R² is selected from C₁-C₆ alkyl, —CH₂-aryl, and —CH₂-heteroaryl, wherein the C₁-C₆ alkyl is unsubstituted or substituted with 1 substituent selected from hydroxy and carboxy, and wherein the aryl or heteroaryl aryl and heteroaryl is independently unsubstituted or substituted with 1 or 2 substituents independently selected from halo, hydroxy, methoxy, amino, and carboxy. In some embodiments, R² is C₁-C₄ alkyl, which is unsubstituted or substituted with 1 substituent selected from hydroxy and carboxy. In some embodiments, R³ is —X—R⁴, wherein X is —C(O)— and R⁴ is selected from C₁-C₆-alkyl, aryl, and heteroaryl, wherein the C₁-C₆ alkyl is unsubstituted or substituted with 1 substituent selected from hydroxy and carboxy, and wherein the aryl or heteroaryl aryl and heteroaryl is independently unsubstituted or substituted with 1 or 2 substituents independently selected from halo, hydroxy, methoxy, amino, and carboxy. In some embodiments, R³ is —X—R⁴, wherein X is —C(O)— and R⁴ is phenyl, wherein the phenyl is unsubstituted or substituted with 1 substituent selected from hydroxy and carboxy.

In some embodiments: R¹ is H; R² is selected from C₁-C₆ alkyl, —CH₂-aryl, and —CH₂-heteroaryl; and R³ is —X—R⁴, wherein X is —C(O)— and R⁴ is selected from C₁-C₆-alkyl, aryl, and heteroaryl; wherein each alkyl is independently unsubstituted or substituted with 1 substituent selected from hydroxy and carboxy; and wherein each aryl and heteroaryl is independently unsubstituted or substituted with 1 or 2 substituents independently selected from halo, hydroxy, methoxy, amino, and carboxy. In some embodiments: R¹ is H: R² is C₁-C₆ alkyl, which is unsubstituted or substituted with 1 substituent selected from hydroxy and carboxy; and R³ is —X—R⁴, wherein X is —C(O)— and R⁴ is phenyl, wherein the phenyl is unsubstituted or substituted with 1 substituent selected from methoxy, hydroxy, and halo.

In some embodiments, the compound of formula (I) is selected from:

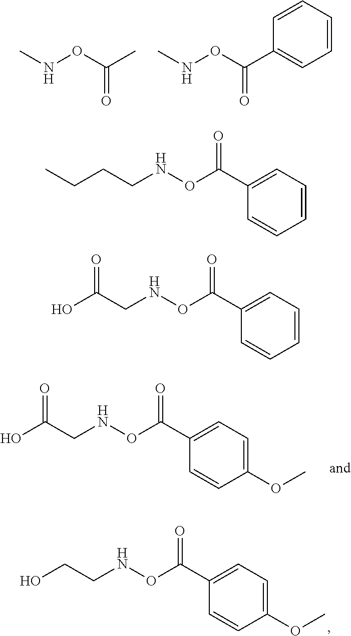

and salts thereof.

In some embodiments, in the compound of formula (I) or the salt thereof:
R¹ is selected from H, C₁-C₆ alkyl, and carboxy-C₁-C₄-alkyl;
R² is selected from C₁-C₆ alkyl, aryl-C₁-C₄-alkyl, heteroaryl-C₁-C₄-alkyl, and —Y—R⁵, wherein Y is selected from a bond, —C(O)—, and —SO₂—, and R⁵ is selected from C₁-C₆ alkyl, aryl, and heteroaryl; and
R³ is H:
wherein each aryl and heteroaryl is independently unsubstituted or substituted with 1 or 2 substituents independently selected from halo, hydroxy, C₁-C₄ alkyl, C₁-C₄ alkoxy, amino, amido, carboxy, and ester; and
wherein each alkyl is unsubstituted or substituted with 1 or 2 substituents independently selected from halo, hydroxy, alkoxy, amino, amido, carboxy, ester, optionally substituted C₃-C₆ cycloalkyl, and optionally substituted 3- to 6-membered heterocyclyl.

In some embodiments, R¹ is H. In some embodiments, R² is selected from C₁-C₆ alkyl, aryl-C₁-C₄-alkyl, heteroaryl-C₁-C₄-alkyl, and —Y—R⁵, wherein Y is selected from a bond, —C(O)—, and —SO₂—, and R⁵ is selected from C₁-C₆ alkyl, aryl, and heteroaryl. In some embodiments, R² is aryl-C₁-C₄-alkyl or heteroaryl-C₁-C₄-alkyl, wherein each aryl and heteroaryl is independently unsubstituted or substituted with 1 or 2 substituents independently selected from halo, hydroxy, C₁-C₄ alkyl, C₁-C₄ alkoxy, amino, amido, carboxy, and ester. In some embodiments, R² is aryl-C₁-C₄-alkyl or heteroaryl-C₁-C₄-alkyl, wherein each aryl and heteroaryl is independently unsubstituted or substituted with 1 or 2 substituents independently selected from halo, hydroxy, methoxy, amino, and carboxy. In some embodiments, R² is aryl-C₁-C₄-alkyl (e.g., benzyl), wherein the aryl is unsubstituted or substituted with 1 or 2 substituents independently selected from halo (e.g., chloro), hydroxy, and methoxy. In some embodiments, R² is heteroaryl-C₁-C₄-alkyl, wherein the heteroaryl is a monocyclic heteroaryl having 1 or 2 heteroatoms independently selected from N, O, and S. In some embodiments, R² is-CH₂-heteroaryl, wherein the heteroaryl is a monocyclic 5- or 6-menmbered heteroaryl having 1 heteroatom selected from N, O, and S.

In some embodiments: R¹ is H: R² is selected from aryl-C₁-C₄-alkyl and heteroaryl-C₁-C₄-alkyl, each of which is independently unsubstituted or substituted with 1 or 2 substituents independently selected from halo, hydroxy, methoxy, amino, and carboxy; and R³ is H. In some embodiments: R¹ is H; R² is aryl-C₁-C₄-alkyl (e.g., benzyl), wherein the aryl is unsubstituted or substituted with 1 or 2 substituents independently selected from halo (e.g., chloro), hydroxy, and methoxy; and R³ is H.

In some embodiments, the compound of formula (I) is selected from:

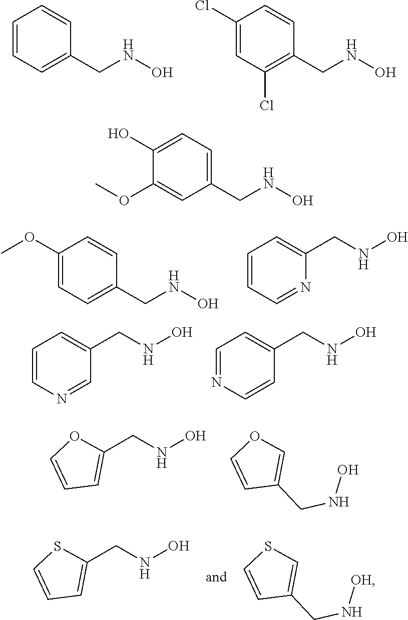

and salts thereof.

In some embodiments, in the compound of formula (I) or the salt thereof:
R¹ and R², together with the nitrogen atom to which they are attached, are taken together to form an optionally substituted 4- to 8-membered ring; and
R³ is H.

In some embodiments, R¹ and R², together with the nitrogen atom to which they are attached, are taken together form a saturated 4- to 8-membered ring that is unsubstituted or substituted with 1 or 2 substituents independently selected from hydroxy, hydroxy-C₁-C₄ alkyl, C₁-C₄-alkoxy, carboxy, carboxy-C₁-C₄ alkyl, and oxo. In some embodiments, R¹ and R², together with the nitrogen atom to which they are attached, are taken together form a saturated 4- to 6-membered ring that is unsubstituted or substituted with 1 or 2 substituents independently selected from hydroxy, hydroxy-C₁-C₄ alkyl, C₁-C₄-alkoxy, carboxy, carboxy-C₁-C₄ alkyl, and oxo. In some embodiments, R¹ and R², together with the nitrogen atom to which they are attached, are taken together form a saturated 5- to 6-membered ring that is unsubstituted or substituted with 1 or 2 substituents independently selected from hydroxy, —CH₂OH, carboxy, —CH₂COOH, and oxo. In some embodiments, R¹ and R², together with the nitrogen atom to which they are attached, are taken together form a pyrrolidine, piperidine, or morpholine ring, each of which is unsubstituted or substituted with 1 or 2 substituents independently selected from hydroxy, —CH₂OH, carboxy, —CH₂COOH, and oxo.

In some embodiments, the compound of formula (I) is selected from:

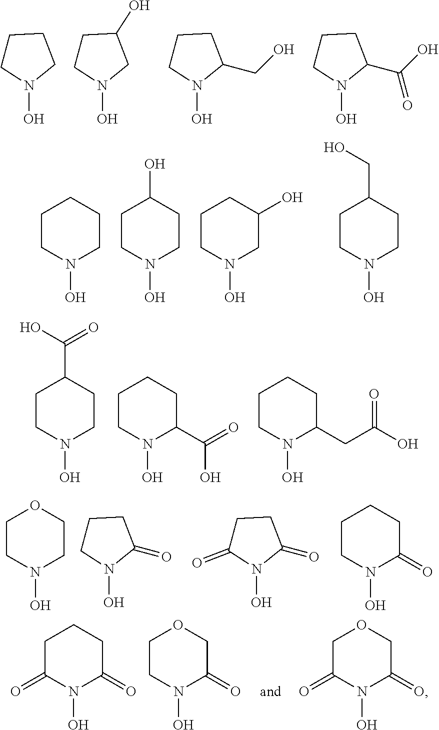

and salts thereof.

In some embodiments, in the compound of formula (I) or the salt thereof:
R¹ is H; and
R² and R³, together with the atoms to which they are attached, are taken together to form an optionally substituted 4- to 8-membered ring.

In some embodiments, R² and R³, together with the atoms to which they are attached, are taken together to form a saturated 4- to 7-membered ring that is unsubstituted or substituted with 1 or 2 substituents independently selected from hydroxy, hydroxy-C₁-C₄ alkyl, C₁-C₄-alkoxy, carboxy, carboxy-C₁-C₄ alkyl, and oxo. In some embodiments, R² and R³, together with the atoms to which they are attached, are taken together form a saturated 5-to 6-membered ring that is unsubstituted or substituted with 1 or 2 substituents independently selected from hydroxy, —CH₂OH, carboxy, —CH₂COOH, and oxo.

In some embodiments, R² and R³, together with the atoms to which they are attached, are taken together to form a saturated 4- to 8-membered ring that is unsubstituted or substituted with 1 or 2 substituents independently selected from hydroxy, hydroxy-C₁-C₄ alkyl, C₁-C₄-alkoxy, carboxy, carboxy-C₁-C₄ alkyl, and oxo. In some embodiments, R² and R³, together with the atoms to which they are attached, are taken together to form a saturated 4- to 7-membered ring that is unsubstituted or substituted with 1 or 2 substituents independently selected from hydroxy, hydroxy-C₁-C₄ alkyl, C₁-C₄-alkoxy, carboxy, carboxy-C₁-C₄ alkyl, and oxo. In some embodiments, R² and R³, together with the atoms to which they are attached, are taken together to form a saturated 4- to 7-membered ring that is unsubstituted or substituted with 1 or 2 substituents independently selected from hydroxy, —CH₂OH, —CH₂CH₂OH, methoxy, carboxy, and oxo. In some embodiments, R² and R³, together with the atoms to which they are attached, are taken together to form a 1,2-oxazetidine, isoxazolidine, 1,2-oxazinane, or 1,2-oxazepane ring, each of which is unsubstituted or substituted with 1 or 2 substituents independently selected from hydroxy, hydroxy-C₁-C₄ alkyl, C₁-C₄-alkoxy, carboxy, carboxy-C₁-C₄ alkyl, and oxo. In some embodiments, R² and R³, together with the atoms to which they are attached, are taken together to form a 1,2-oxazetidine, isoxazolidine, 1,2-oxazinane, or 1,2-oxazepane ring, each of which is unsubstituted or substituted with 1 substituent selected from hydroxy, —CH₂OH, —CH₂CH₂OH, methoxy, carboxy, and oxo.

In some embodiments, the compound of formula (I) is selected from:

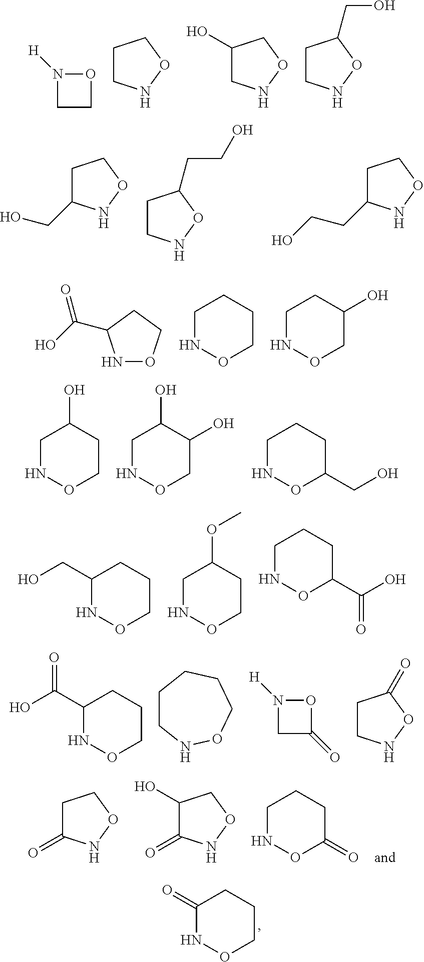

and salts thereof.

In some embodiments, the compound of formula (I) is selected from:

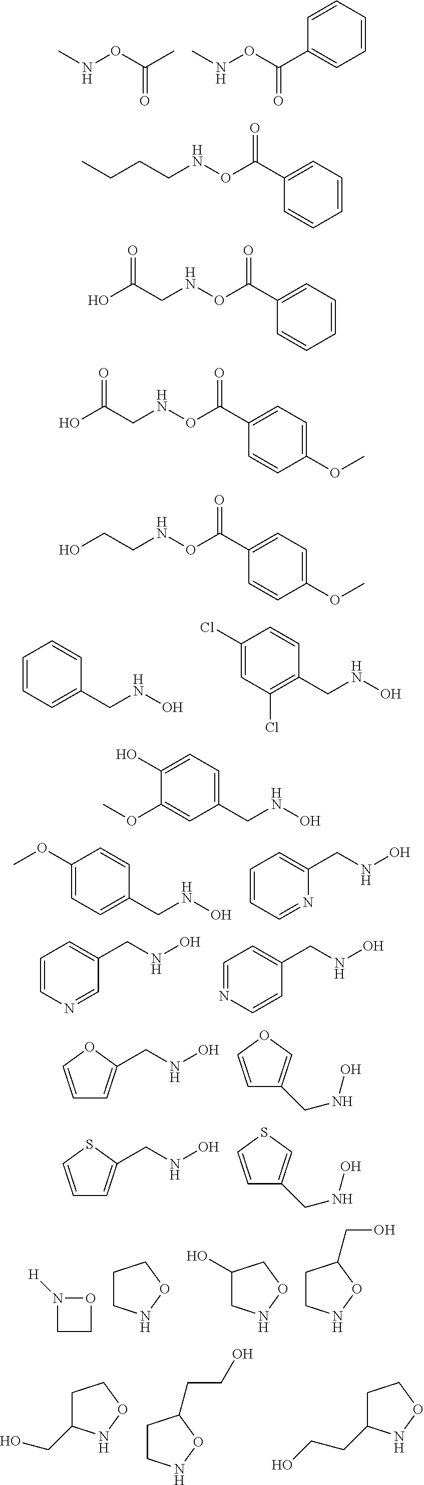

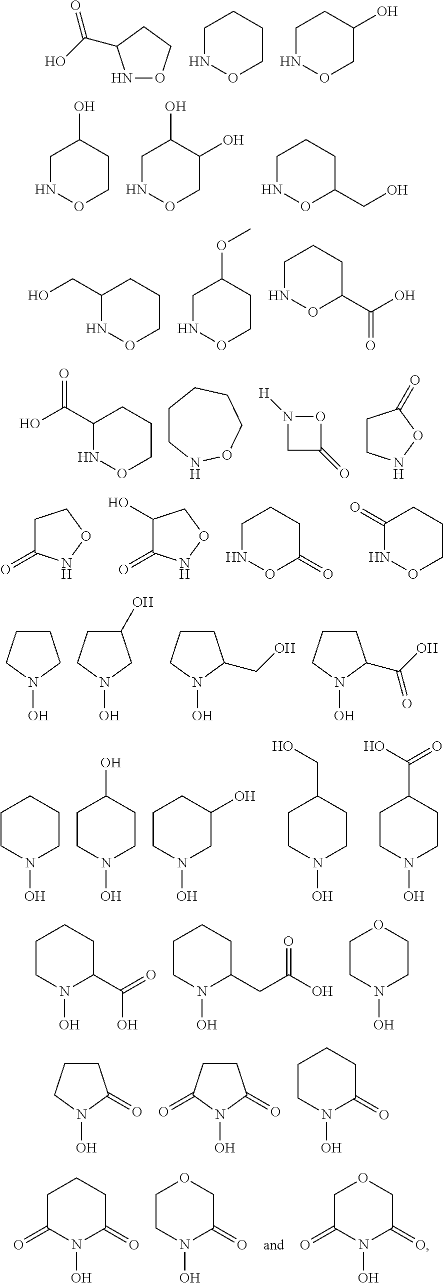

and salts thereof.

In some embodiments, the compound is selected from:

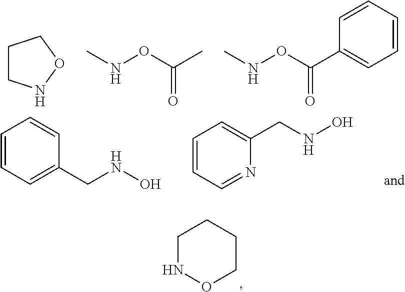

and salts thereof.

In some embodiments, the compound of formula (I) is in the form of a salt. In some embodiments, the compound of formula (I) is in the form of an acid addition salt. An acid addition salt can be formed with an inorganic acid or an organic acid. Inorganic acids from which salts can be derived include, for example, hydrochloric acid, hydrobromic acid, sulfuric acid, nitric acid, phosphoric acid, and the like. Organic acids from which salts can be derived include, for example, acetic acid, trifluoroacetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. In some embodiments, the compound of formula (I) is in the form of a hydrochloric acid salt.

Unless otherwise stated, structures depicted herein are also meant to include geometric (or conformational) forms of the structure: for example, the R and S configurations for each asymmetric center, Z and E double bond isomers, and Z and E conformational isomers. Therefore, single stereochemical isomers as well as enantiomeric, diastereomeric, and geometric (or conformational) mixtures of the disclosed compounds are within the scope of the disclosure. Unless otherwise stated, all tautomeric forms of the compounds described herein are within the scope of the disclosure.

Additionally, unless otherwise stated, structures depicted herein are also meant to include compounds that differ only in the presence of one or more isotopically enriched atoms. For example, compounds having the disclosed structures including the replacement of hydrogen by deuterium or tritium, or the replacement of a carbon by a ¹³C or ¹⁴C enriched carbon, are within the scope of this disclosure.

The compounds of formula (I) are useful for decrosslinking a formaldehyde cross-linked biological sample, such as an FFPE tissue sample. Accordingly, in some embodiments, disclosed herein is a composition comprising a formaldehyde cross-linked biological sample (e.g., an FFPE sample), and a compound of formula (I) as described herein. In some embodiments, the composition comprises more than one different compound of formula (I) (i.e., the composition comprises a mixture of two or more different compounds of formula (I)).

In some embodiments, the composition comprising the compound(s) of formula (I) is in the form of a solution, such as an aqueous solution. In some embodiments, the composition further comprises a buffer. For example, in some embodiments, the buffer is selected from tris (hydroxymethyl) aminomethane (Tris), 2-(N-morpholino) ethanesulfonic acid (MES), 4-(2-hydroxyethyl)-1-piperazineethanesulfonic acid (HEPES), phosphate-buffered saline, glycine, and citrate. Without wishing to be limited by theory, when used in the decrosslinking methods described herein, certain buffers may aid in sequestering/quenching formaldehyde from the decrosslinked FFPE in the sample.

### Methods

Disclosed herein are methods of decrosslinking a formaldehyde cross-linked biological sample, comprising contacting the sample with an effective amount of a compound of formula (I), or a salt thereof. The disclosed methods can use any compound of formula (I) described herein, such as those specifically exemplified above, or any combination of at least two compounds of formula (I), or salts thereof. (i.e., at least two different compounds of formula (I), or salts thereof).

In embodiments in which two or more different compounds of formula (I) are used, they can be added to the sample either simultaneously (e.g., in the same composition or simultaneously from separate compositions) or sequentially.

In some embodiments, the sample is a formalin-fixed paraffin embedded tissue sample. In some embodiments in which the sample is an FFPE sample, the methods further comprise a step of deparaffinizing the sample prior contacting the sample with the compound of formula (I) (or the salt thereof). Deparaffinizing can be conducted according to standard methods. A common deparaffinization protocol includes incubating the sample with mineral oil at about 80° C. for 1-3 minutes, and optionally repeating the mineral oil treatment one or more additional times to complete the paraffin removal process. Other methods of deparaffinizing samples may also be used, such as treatment with xylenes. In some embodiments, the FFPE sample is not deparaffinized prior to any further treatment steps.

The methods may further include a step of treating the sample with a protease prior to contacting the sample with the compound of formula (I) (or the salt thereof). When detecting nucleic acids, the protease treatment can eliminate contaminating proteins, such as DNases and RNases. In some embodiments, the protease is selected from proteinase K. trypsin, LysC, ProAlanase, and pepsin. In some embodiments, the protease is proteinase K. The protease treatment can be conducted for an appropriate time and at an appropriate temperature, e.g., about 50 to 60° C. for about 30 minutes to about 18 hours.

The step of contacting the sample with the compound of formula (I) (or salt thereof) can be conducted for a period of time sufficient to decrosslink the sample. In some embodiments, the contacting step is conducted for about 5 minutes to about 120 minutes, or about 20 minutes to about 40) minutes. In some embodiments, the contacting step is conducted for about 5 minutes, about 10 minutes, about 15 minutes, about 20 minutes, about 25 minutes, about 30) minutes, about 35 minutes, about 40) minutes, about 45 minutes, about 50) minutes, about 55 minutes, about 60) minutes, about 70) minutes, about 80 minutes, about 90 minutes, about 100) minutes, about 110 minutes, or about 120 minutes.

The step of contacting the sample with the compound of formula (I) (or salt thereof) can be conducted at a temperature that is suitable for decrosslinking the sample. In some embodiments, the contacting step is conducted at a temperature of about 20° C. to about 100° C., or about 50° C. to about 80° C. In some embodiments, the contacting step is conducted at ambient (i.e., room) temperature. In some embodiments, the contacting step is conducted at a temperature of about 20° C. about 25° C. about 30° C. about 35° C. about 40° C. about 45° C. about 50° C. about 55° C. about 60° C. about 65° C. about 70° C. about 75° C. about 80° C. about 85° C. about 90° C. or about 95° C.

The step of contacting the sample with the compound of formula (I) (or salt thereof) can be conducted by contacting the sample with a solution of the compound of formula (I) (or salt thereof), e.g., an aqueous solution comprising the compound of formula (I) (or salt thereof). In such embodiments, the resulting solution has a pH upon contacting the sample of about 4 to about 8.5, or about 4.5 to about 6.5. In some embodiments, the pH of the aqueous solution is about 4.0, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0), about 7.1, about 7.2, about 7.3, about 7.4, about 7.5, about 7.6, about 7.7, about 7.8, about 7.9, about 8.0, about 8.1, about 8.2, about 8.3, about 8.4, or about 8.5.

In some embodiments, the aqueous solution comprises the compound of formula (I) (or salt thereof) at a concentration of about 1 mM to about 120 mM, or about 5 mM to about 100 mM. In some embodiments, the aqueous solution comprises the compound of formula (I) (or salt thereof) at a concentration of about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30) mM, about 31 mM, about 32 mM, about 33 mM, about 34 mM, about 35 mM, about 36 mM, about 37 mM, about 38 mM, about 39 mM, about 40 mM, about 41 mM, about 42 mM, about 43 mM, about 44 mM, about 45 mM, about 46 mM, about 47 mM, about 48 mM, about 49 mM, about 50 mM, about 51 mM, about 52 mM, about 53 mM, about 54 mM, about 55 mM, about 56 mM, about 57 mM, about 58 mM, about 59 mM, about 60 mM, about 61 mM, about 62 mM, about 63 mM, about 64 mM, about 65 mM, about 66 mM, about 67 mM, about 68 mM, about 69 mM, about 70 mM, about 71 mM, about 72 mM, about 73 mM, about 74 mM, about 75 mM, about 76 mM, about 77 mM, about 78 mM, about 79 mM, about 80 mM, about 81 mM, about 82 mM, about 83 mM, about 84 mM, about 85 mM, about 86 mM, about 87 mM, about 88 mM, about 89 mM, about 90 mM, about 91 mM, about 92 mM, about 93 mM, about 94 mM, about 95 mM, about 96 mM, about 97 mM, about 98 mM, about 99 mM, about 100 mM, about 101 mM, about 102 mM, about 103 mM, about 104 mM, about 105 mM, about 106 mM, about 107 mM, about 108 mM, about 109 mM, about 110 mM, about 111 mM, about 112 mM, about 113 mM, about 114 mM, about 115 mM, about 116 mM, about 117 mM, about 118 mM, about 119 mM, or about 120 mM. In embodiments in which more than one compound of formula (I) is used, each one can be individually present at any of the indicated concentrations.

The methods can efficiently decrosslink cross-linked biological samples, such as FFPE tissue samples, without sacrificing quality of the biological materials contained within the samples, such as nucleic acids. In some embodiments, the methods further comprise a step of extracting one or more components from the sample after the contacting step. In some embodiments, the one or more components are selected from nucleic acids and proteins. In some embodiments, the nucleic acids are DNA. In some embodiments, the nucleic acids are RNA. In some embodiments, the one or more components are proteins.

The methods may further include a method of detecting one or more components following its extraction from the sample. For example, in embodiments in which a nucleic acid is extracted from the sample, the nucleic acids can be detected by, for example, nucleotide sequencing or sequence-specific hybridization. In some embodiments, the methods further comprise a step of amplifying one or more nucleic acids from the sample. Any time of amplification may be used, including polymerase chain reaction (PCR), quantitative PCR, real time PCR, hot start PCR, single cell PCR, nested PCR, in situ colony PCR, digital PCR (dPCR). Droplet Digital™ PCR (ddPCR), emulsion PCR, ligase chain reaction (LCR), transcription based amplification system (TAS), nucleic acid sequence-based amplification (NASBA), strand displacement amplification (SDA), rolling circle amplification (RCA), and hyper-branched RCA (HRCA). In some embodiments, the nucleic acid amplification reaction is a multiplex nucleic acid amplification reaction. In some embodiments, the sequencing analysis comprises fragment analysis and/or Sanger sequencing analysis, or Next Generation Sequencing (NGS) analysis.

Other characterization methods include dye binding, absorbance, and enzymatic digestion. For example, dye binding assays use fluorescent dyes specific to nucleic acids and a fluorometer for quantification. Absorbance assays are spectrophotometric assays that rely on nucleic acid's natural light absorbing properties to determine concentration and purity. Enzymatic digestions include applications in which FFPE nucleic acids are digested with enzymes to improve quality or as an intermediate step for further experimentation. For example. Uracil-DNA Glycosylase can improve FFPE DNA quality by excising uracil nucleobases that have originated from cytosine deamination, while SI Nuclease digests single-stranded DNA and can improve FFPE DNA quality by increasing proportion of double-stranded DNA and reducing artifacts that originate from single-stranded DNA templates.

In some embodiments, when proteins are extracted from the sample, the proteins can be detected by a variety of methods. In some embodiments, the proteins can be detected by mass spectrometry. Suitable mass spectrometry methods include electrospray ionization, and matrix-assisted laser desorption/ionization (MALDI) (e.g., MALDI time of flight (MALDI-TOF) methods. In some embodiments, the detecting step comprises a colorimetric assay (e.g., a BCA assay or Bradford assay), fluorescence spectroscopy (e.g., using dye binding such as a fluorimetric peptide assay, or by detecting fluorescence from tryptophan residues in the protein), UV-vis spectrophotometry (e.g., absorbance at 280 nm), electrophoresis (e.g., SDS-PAGE, which may be followed by staining such as silver staining) or an immunoassay (e.g., Western blotting or immunohistochemistry).

### Systems and Kits

Also disclosed herein are systems and kits for decrosslinking a formaldehyde cross-linked biological sample. The systems and kits comprise a compound of formula (I), or a salt thereof, such as any compound of formula (I) described herein, including those specifically exemplified above. In some embodiments, the system or kit comprises one or more compounds of formula (I) (or salts thereof), such as a combination of two or more compounds of formula (I) (or salts thereof).

In some embodiments, the system or kit includes the compound either alone or in a solvent such as water, DMSO, or a buffer. When the compound is provided in the absence of a solvent, the system or kit may further include the solvent in which the compound can be dissolved. The system or kit may further comprise one or more reagents used to carry out a decrosslinking reaction and/or a downstream assay such as a detection method described herein. The system or kit may further comprise instructions, such as instructions for carrying out a decrosslinking reaction of a cross-linked biological sample, or instructions for carrying out a downstream assay such as a detection method.

In some embodiments, the system or kit further comprises other compounds that are used in the methods disclosed herein. For example, in some embodiments, the system or kit further comprises a protease, such as proteinase K. In some embodiments, the system or kit further comprises a compound for conducting a deparaffinization step, such as mineral oil or xylenes.

### EXAMPLES

### Example 1

### Decrosslinking Compound Screening

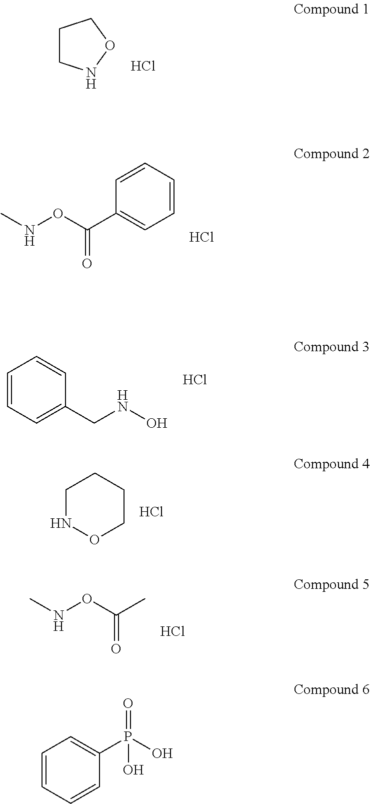

Candidate decrosslinking catalyst compounds, Compounds 1-5, were screened by adding them to a FFPE tissue lysate after a Proteinase K (ProK) digestion (56° C. for 30 minutes), followed by decrosslinking at 80° C. (Compound 6 was tested separately and results are presented below.) For a positive control, decrosslinking was conducted at 80° C. for 4 hours, and as a negative control, decrosslinking was conducted at 80° C. for 30 minutes. Candidate decrosslinking compounds, at a concentration of either 20 mM or 100 mM, were placed into lysates buffered at a pH of 7.4, 8.4, or 9.6 and were incubated with the samples at 80° C. for 30 minutes. Decrosslinked nucleic acids were purified using the MaxwellR RSC DNA FFPE Kit (Promega Cat. No. AS1450) on the Maxwell® RSC 48 Instrument (Promega Cat. No. AS8500) according to the manufacturer's protocol. After extraction of nucleic acids, DNA recovery was determined by qPCR analysis using primers specific to RNaseP (“R”) and telomerase reverse transcriptase (“T”), resulting in 102 bp and 164 bp amplicons, respectively. Screens were conducted with cell pellet artificial FFPE derived from different tissue types and immortalized cell lines. Results are shown in Tables 1 and 2, each of which shows DNA yield (ng) (top) and relative DNA yields normalized against the negative control (bottom). As can be seen, decrosslinking compounds disclosed herein show similar recovery yield at 30 minutes compared with incubation without the decrosslinking compounds at 4 hours.

TABLE 1

| | Decrosslink | Lysis | Test cpd. | Hydroxylamine | | Compound 1 | | Compound 2 | |
|---|---|---|---|---|---|---|---|---|---|
| | Time (hr) | buffer pH | conc. (mM) | R | T | R | T | R | T |
| Pos. Control | 4 | 7.4 | 0 | 383.4 | 76.2 | 755.7 | 1099.6 | 846.9 | 1242.4 |
| Neg. Control | 0.5 | 7.4 | 0 | 15.4 | 2.5 | 50.9 | 20.7 | 121.6 | 161.2 |
| | 0.5 | 7.4 | 20 | 24.6 | 5.1 | 597.3 | 921.4 | 491.9 | 1186.1 |
| | 0.5 | 8.4 | 20 | 23.5 | 5.5 | 514.1 | 1443.9 | 90.8 | 63.1 |
| | 0.5 | 9.6 | 20 | 21.9 | 8.7 | 89.2 | 37.2 | 0.1 | 0.0 |
| | 0.5 | 7.4 | 100 | 122.9 | 54.7 | 14.8 | 0.1 | 0.0 | 0.0 |
| | 0.5 | 8.4 | 100 | 73.1 | 8.4 | 79.9 | 13.5 | 0.0 | 0.0 |
| | 0.5 | 9.6 | 100 | 36.2 | 8.1 | 215.5 | 180.8 | 0.0 | 0.0 |
| Pos. Control | 4 | 7.4 | 0 | 25.0 | 30.0 | 14.8 | 53.1 | 7.0 | 7.7 |
| Neg. Control | 0.5 | 7.4 | 0 | 1.0 | 1.0 | 1.0 | 1.0 | 1.0 | 1.0 |
| | 0.5 | 7.4 | 20 | 1.6 | 2.0 | 11.7 | 44.5 | 4.0 | 7.4 |
| | 0.5 | 8.4 | 20 | 1.5 | 2.2 | 10.1 | 69.7 | 0.7 | 0.4 |
| | 0.5 | 9.6 | 20 | 1.4 | 3.4 | 1.8 | 1.8 | 0.0 | 0.0 |
| | 0.5 | 7.4 | 100 | 8.0 | 21.5 | 0.3 | 0.0 | 0.0 | 0.0 |
| | 0.5 | 8.4 | 100 | 4.8 | 3.3 | 1.6 | 0.7 | 0.0 | 0.0 |
| | 0.5 | 9.6 | 100 | 2.4 | 3.2 | 4.2 | 8.7 | 0.0 | 0.0 |

| | | | |
|---|---|---|---|
| Tissue: | Kidney | Breast | Breast |
| Cell Line: | 293T | MCF7 | MCF7 |
| Block: | 201022A | 191105A | 191105A |
| Replicates: | two | three | three |

TABLE 2

| | Decrosslink | Lysis | Test cpd. | Compound 3 | | Compound 4 | | Compound 5 | |
|---|---|---|---|---|---|---|---|---|---|
| | Time (hr) | buffer pH | conc. (mM) | R | T | R | T | R | T |
| Pos. Control | 4 | 7.4 | 0 | 408.4 | 1137.1 | 493.0 | 1746.2 | 778.5 | 1032.7 |
| Neg. Control | 0.5 | 7.4 | 0 | 52.5 | 36.0 | 31.2 | 29.2 | 34.6 | 13.6 |
| | 0.5 | 7.4 | 20 | 379.5 | 1142.6 | 400.6 | 1361.0 | 563.9 | 1217.1 |
| | 0.5 | 8.4 | 20 | 153.3 | 280.0 | 294.6 | 843.6 | 148.8 | 71.0 |
| | 0.5 | 9.6 | 20 | 65.5 | 85.3 | 63.4 | 155.1 | 69.3 | 34.3 |
| | 0.5 | 7.4 | 100 | 34.2 | 10.9 | 8.1 | 0.6 | 0.0 | 0.0 |
| | 0.5 | 8.4 | 100 | 112.5 | 104.4 | 56.1 | 29.7 | 54.9 | 0.0 |
| | 0.5 | 9.6 | 100 | 214.4 | 445.3 | 97.4 | 275.9 | 165.9 | 140.8 |
| Pos. Control | 4 | 7.4 | 0 | 7.8 | 31.6 | 15.8 | 59.8 | 22.5 | 76.2 |
| Neg. Control | 0.5 | 7.4 | 0 | 1.0 | 1.0 | 1.0 | 1.0 | 1.0 | 1.0 |
| | 0.5 | 7.4 | 20 | 7.2 | 31.7 | 12.9 | 46.6 | 16.3 | 89.8 |
| | 0.5 | 8.4 | 20 | 2.9 | 7.8 | 9.5 | 28.9 | 4.3 | 5.2 |
| | 0.5 | 9.6 | 20 | 1.2 | 2.4 | 2.0 | 5.3 | 2.0 | 2.5 |
| | 0.5 | 7.4 | 100 | 0.7 | 0.3 | 0.3 | 0.0 | 0.0 | 0.0 |
| | 0.5 | 8.4 | 100 | 2.1 | 2.9 | 2.1 | 1.0 | 1.6 | 0.0 |
| | 0.5 | 9.6 | 100 | 4.1 | 12.4 | 3.1 | 9.5 | 4.8 | 10.4 |

| | | | |
|---|---|---|---|
| Tissue: | Breast | Breast | Breast |
| Cell Line: | MCF7 | MCF7 | MCF7 |
| Block: | 191014A | 210615A | 191105A |
| Replicates: | three | three | three |

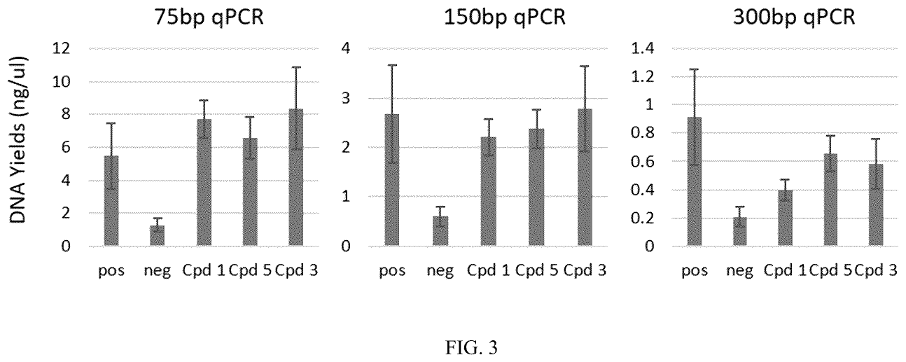

The quality of DNA decrosslinked by these leading candidate was further accessed by testing on FFPE tissues from human donors using Next Generation Sequencing (NGS). Healthy colon FFPE donor tissue underwent rapid preprocessing using Compounds 1, 3, and 5 (Cpd. 1, Cpd. 3, and Cpd. 5, respectively) with decrosslinking carried out at 80° C. for 30 minutes, along with positive controls (80° C. for 4 hours) and negative controls (80° C. for 30 minutes). DNA was then purified on the MaxwellR: RSC 48 instrument (Promega Corp). DNA quantity, and fragment sizes were assessed using Promega's ProNexR: DNA QC Assay, which amplifies 75 bp. 150 bp, and 300 bp qPCR amplicons. Amplifiable DNA quantity and fragment sizes from samples rapidly decrosslinked with Cpd 1, Cpd 5, and Cpd 3 reactions were comparable with positive control samples (FIG. 3). Averages of three purification replicates were plotted with error bars representing standard deviation.

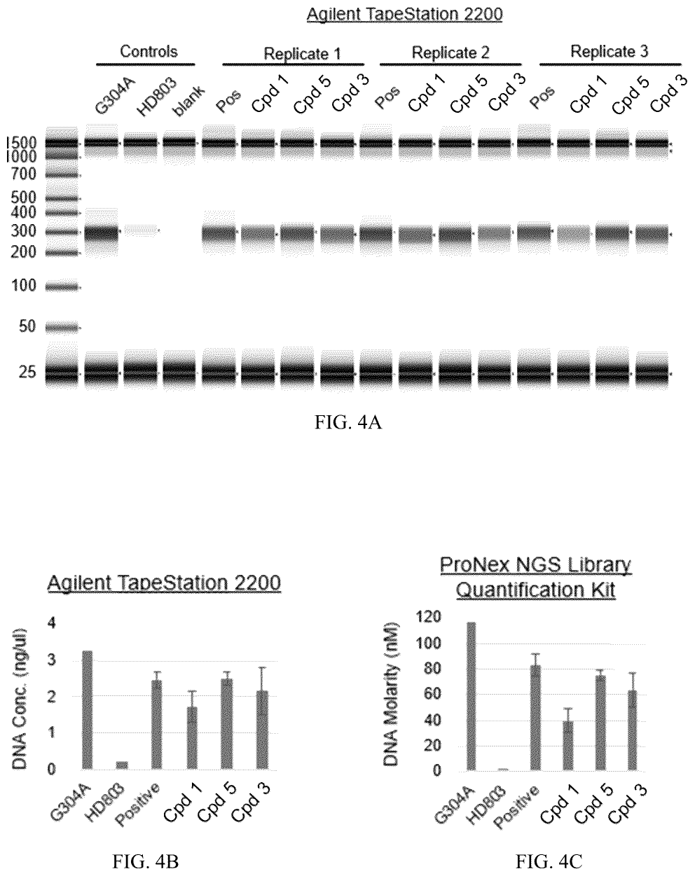

NGS libraries were constructed using Illumina's AmpliSeq for Cancer HotSpot Panel v2. FIG. 4A shows molecular weight of libraries as determined by electrophoresis with Agilent TapeStation. FIG. 4B shows quantification of electrophoresed libraries. FIG. 4C shows quantity of libraries as determined by qPCR with ProNex′R: NGS Library Quantification Kit. G304A is high quality human gDNA pooled from multiple donors (Promega Cat. No. G3041). HD803 is formalin-damaged human gDNA pooled from multiple immortalized cell lines for use as a Multiplex Reference Standard (Horizon, HD803). NGS libraries from rapid decrosslinking reactions were of expected size and sufficient quantity for sequencing.

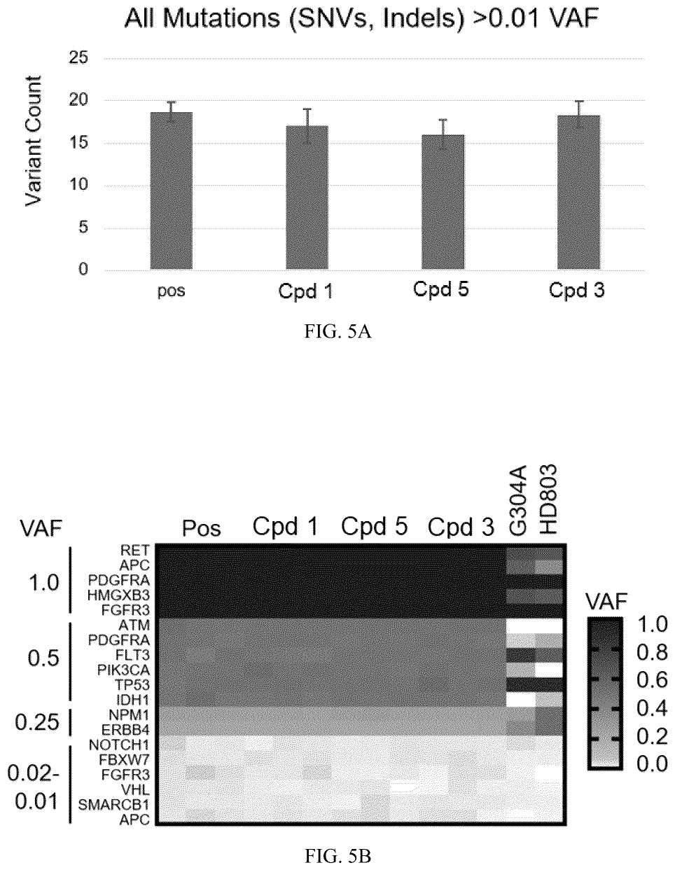

Libraries were sequenced using paired-end 150-cycle run on an Illumina MiSeq. Sequeence Analysis Viewer and FastQC was used to assess sequencing performance. Error rate, % Q30, cluster density, cluster % PF, Phas/Prephas (%) were all within expected ranges (data not shown). Bioinformatic analyses was performed with llumina's BaseSpace DNA Amplicon App. Single-nucleotide variants (SNVs) and insert-deletions (Indels) with variant allele frequencies (VAF) above 1% were called. Total counts were comparable between positive control and rapid decrosslinking catalyst purifications (FIG. 5A). VAFs of mutations identified in the positive control were found to be highly concordant with catalyst purifications (FIG. 5B). This indicates that rapid decrosslinked DNA maintains high sequence quality, allowing for NGS to be used to make VAF calls near the limit-of-detection.

### Example 2

### Decrosslinking Time Course

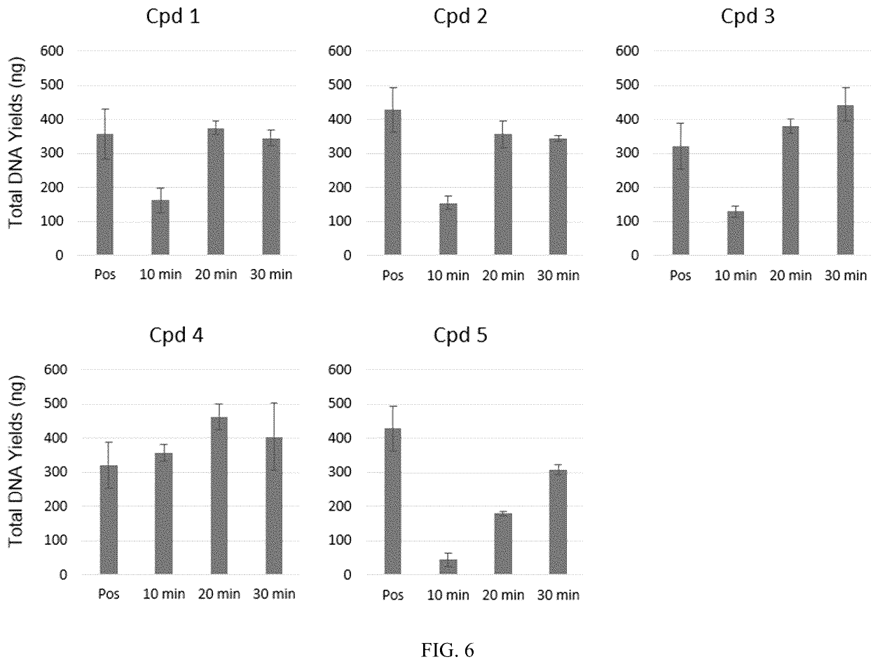

A time course experiment was conducted to determine the minimal decrosslinking time needed for complete decrosslinking with catalyst compounds (FIG. 6). Cell pellet artificial FFPE (Amsbio LLC) were decrosslinked with Cpd 1-5 at 80° C. for 10, 20, and 30 minutes. Amplifiable DNA yields were accessed using a PrimeTime qPCR Assay targeting a 102 bp amplicon in the RPPHI gene (IDT). Minimal decrosslinking times ranged from 10 minutes (Cpd 4), 20 minutes (Cpd 1, Cpd 2, Cpd 3), and 30 minutes (Cpd 5). DNA yields were compared to a positive control (Pos) decrosslinked at 80° C. for 4 hours without catalysts.

### Example 3 pH Optimization

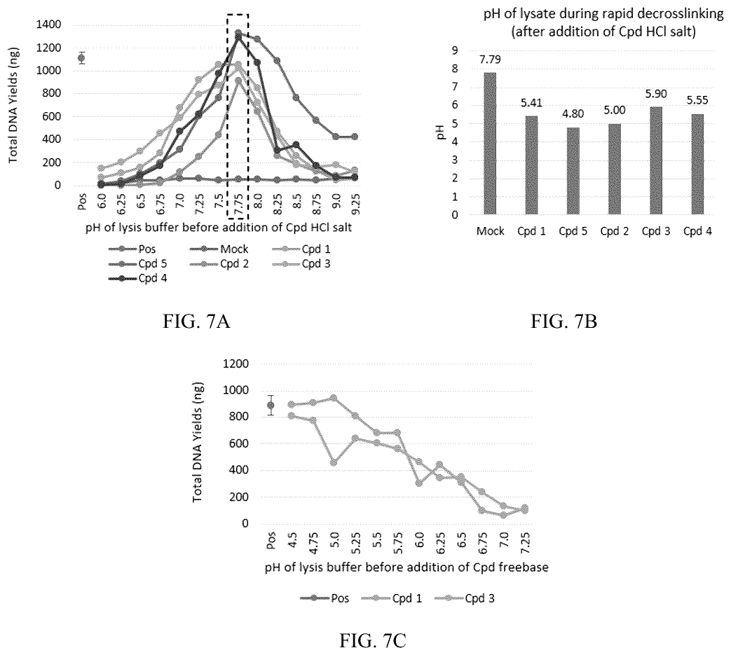

To determine the optimal pH for rapid decrosslinking, cell pellet artificial FFPE (Amsbio LLC) were decrosslinked at 80° C. for 30 minutes with 20 mM of Cpd 1-5 HCl salts in a range of pH lysis buffers (FIG. 7A). Amplifiable DNA yields were accessed using a PrimeTime qPCR Assay targeting a 164 bp amplicon in the TERT gene (IDT). DNA yields were compared to a positive control (Pos) decrosslinked in pH 8 lysis buffer at 80° C. for 4 hours without catalysts, and a negative control (mock) decrosslinked in a range of pH lysis buffers at 80° C. for 30 minutes without catalysts. To determine the working pH of the lysate during rapid decrosslinking, 20 mM of Cpd HCl salts were added to lysis buffer pH 7.75 (optimum found in FIG. 7A). Optimal pH fell between 4.80-5.90 (FIG. 7B). To confirm the pH requirement can be achieved through the buffer, an experiment was performed using catalyst freebases (FIG. 7C). After Proteinase K digestion, pH of lysates were lowered with pre-determined amounts of HCl to create a range of pH lysis buffers. 20 mM of freebase Cpd 1 and Cpd 3 were added to the lysate and decrosslinked for 30 minutes at 80° C. Amplifiable DNA yields were accessed using a PrimeTime qPCR Assay targeting a 164 bp amplicon in the TERT gene (IDT). DNA yields peaked in buffers with pH 4.5-6.0.

### Example 4

### Catalyst Concentration Optimization

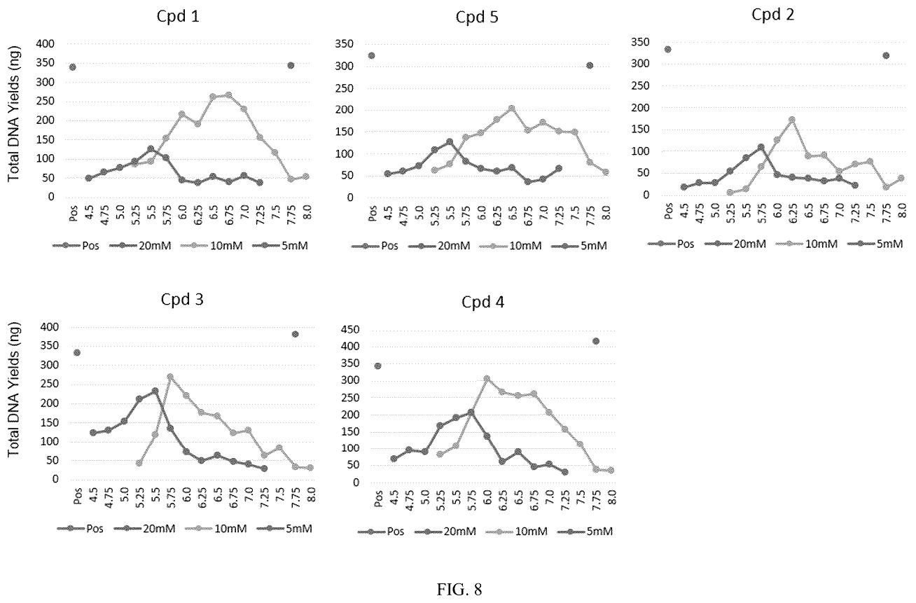

Optimal concentration of decrosslinking catalyst compounds were determined by adding 20 mM, 10 mM, and 5 mM of Cpd HCl 1-5 and decrosslinking at 80° C. for 30 minutes (FIG. 8). A range of pH lysis buffers were used to accommodate for less HCl (from the Cpd HCl salt) being added. Amplifiable DNA yields were accessed using a PrimeTime qPCR Assay targeting a 102 bp amplicon in the RPPHI gene (IDT). DNA yields were compared to a positive control (Pos) decrosslinked in pH 8 lysis buffer at 80° C. for 4 hours without catalysts. Amplifiable DNA yields were found to be concentration dependent.

### Example 5

### Catalyst-Decrosslinked FFPE DNA Purified From a Variety of Human Tissues and Assessed by Amplification, Dye-Binding, and Absorbance

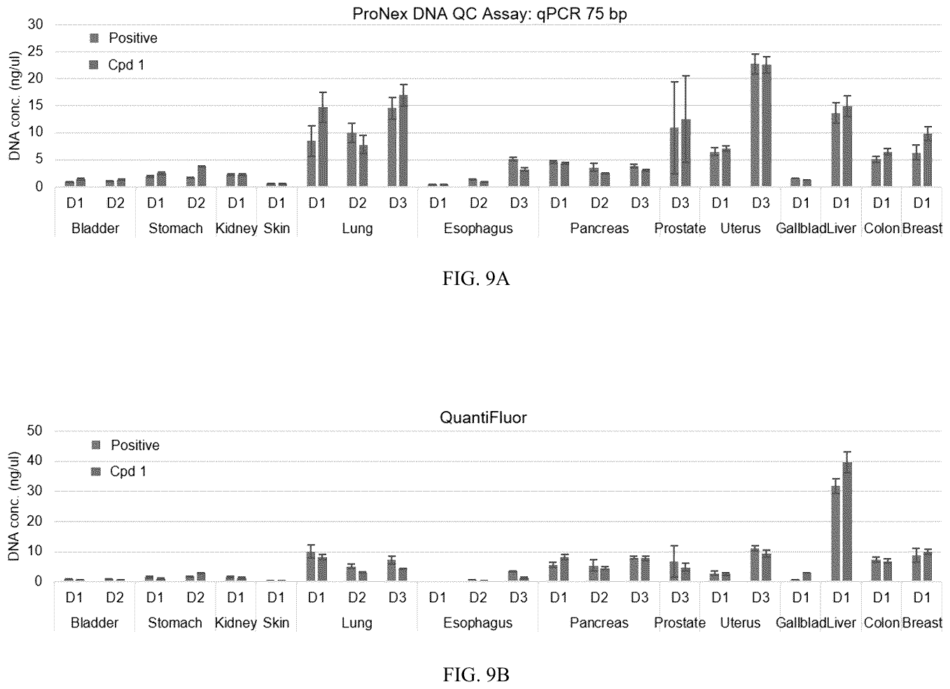

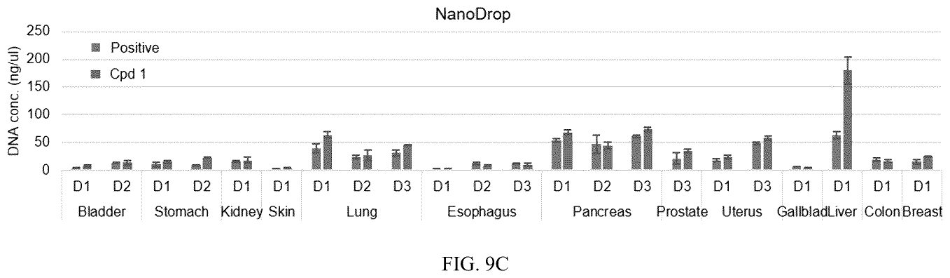

Catalysts were tested in 13 human FFPE tissues (FIGS. 9A-9C: bladder, stomach, kidney, skin, lung, esophagus, pancreas, prostate, uterus, gallbladder, liver, colon, and breast). 30 mM compound 1 (Cpd 1) was added to each FFPE lysate and decrosslinked at 80° C. for 30 minutes (Cpd 1) and compared to a mock no compound treatment decrosslinked at 80° C. for 4 hours (Positive). One to three donors per tissue, and two to four purification replicates per donor were performed. DNA yields were assessed by RT-qPCR using Promega ProNexR DNA QC Assay (Cat. No. NG1002) (FIG. 9A), fluorescent dsDNA dye using Promega QuantiFluor R: dsDNA System (Cat. No. E2671) (FIG. 9B), and absorbance using a ThermoFisher NanoDrop (FIG. 9C).

### Example 6

### Overview of FFPE Protein Extraction Workflow

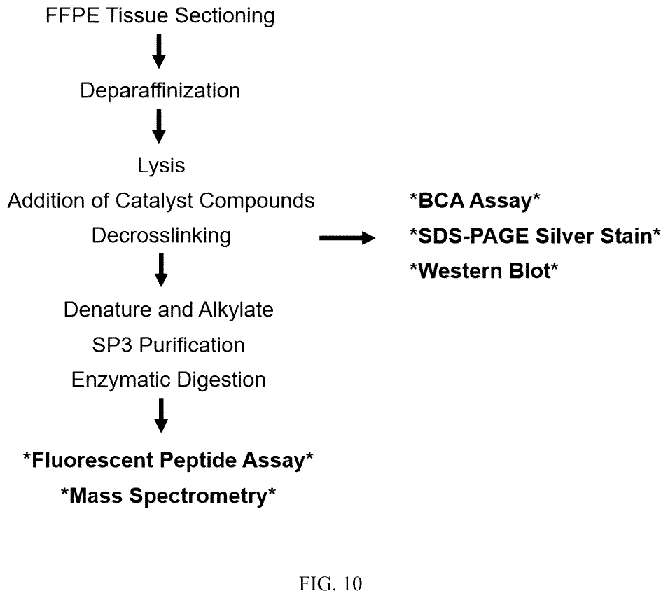

A workflow for the extraction of protein from FFPE was developed as outlined in FIG. 10. Briefly, human colon and human lung FFPE blocks were sectioned at 20 μm with a microtome. Sections were deparaffinized by washing in 100% Xylene followed by washing in 100% Ethanol. Deparaffinized FFPE tissue was resuspended in lysis buffer containing 50 mM HEPES pH 8.0, 50 mM NaCl, 2 mM MgCl₂, 2% SDS, and 1% Glycerol. The resulting lysate was sonicated for a total run time of 10 minutes, using 10 seconds on/10 seconds off pulses, with a microtip probe receiving a total of ˜10,000 J. Benzonase was added to a concentration of 100 units/ml and the lysate was incubated at 37° C. for 30 minutes. The lysates either received decrosslinking catalysts or mock treatments (H₂O or DMSO), then underwent a decrosslinking incubation at various temperatures (65° C.-95° C.) and times (10) minutes-8 hours). The resulting crude protein extracts were analyzed by the Pierce BCA Protein Assay (ThermoFisher), SDS-PAGE Silver Stain, or western blot analysis. Select crude protein extracts were further reduced and alkylated with 10 mM TCEP and 40 mM Chloroacetamide and incubating at 37° C. for 30 minutes. Protein was purified using an SP3 workflow (Hughes et al. Nat Protoc. 2019: 14 (1): 68-85). Briefly, each protein sample received 250 μg MagSil beads and 80% acetonitrile. Protein was bound and washed with 70% ethanol followed by 100% acetonitrile. Protein was resuspended in 30 μl of proteolysis buffer (200 mM HEPES pH 8.0, Trypsin/Lys-C 1:25 E/S ratio) and incubated at 37° C. for 18 hours. The digested peptides were then quantitated using the Pierce Quantitative Fluorometric Peptide Assay (ThermoScientific). Normalized peptide amounts were then subjected to LC-MS/MS using a 1-hour gradient (0-36% B (80% Acetonitrile)) using DIA-MS detection on an Exploris 240 Orbitrap Mass Spectrometer (ThermoScientific).

### Example 7

### FFPE Protein Requires a Heated Decrosslinking Incubation

In this example, the role of the decrosslinking incubation for FFPE protein extractions was first characterized. FFPE was subjected to increasing decrosslinking incubation times ranging from 0 to 120 minutes at a temperature of 90° C. and tested in triplicate. The resulting lysates were centrifuged to separate soluble and pelleted insoluble protein fractions. Soluble and insoluble protein fractions were analyzed using SDS-PAGE followed by silver staining

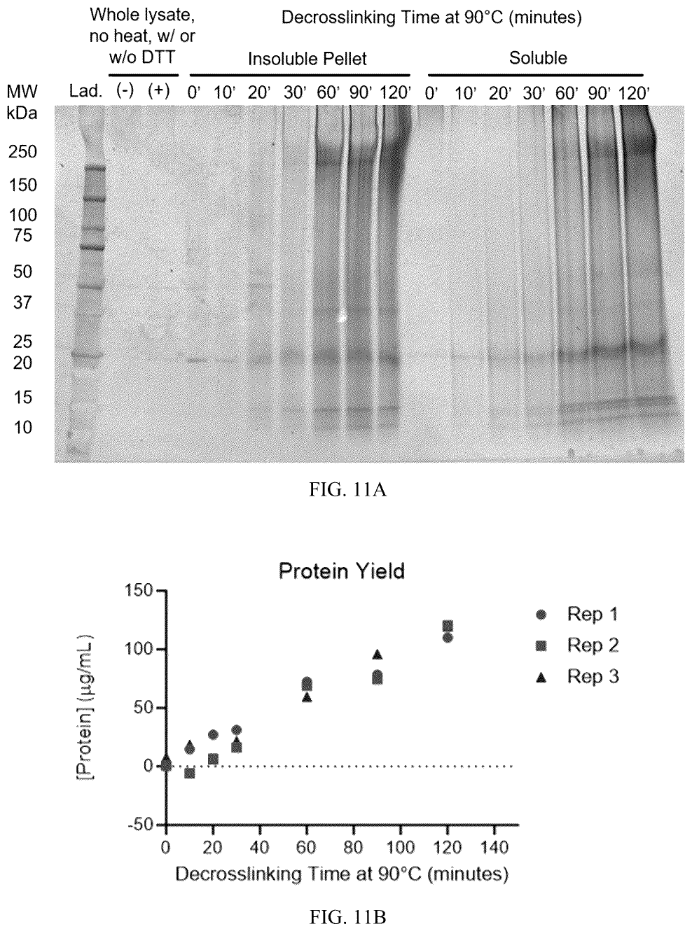

(FIG. 11A). Longer decrosslinking times resulted in greater yields of mobile protein from both soluble and insoluble fractions. A role for denaturing agents in decrosslinking was also tested using 20 mM DTT, but no effect on mobile protein was observed (FIG. 11A). The Pierce BCA Assay was used to further quantitate soluble protein (FIG. 11B). Yields ranged from <5 μg/ml with no decrosslinking incubation up to 120 μg/ml with 120 minutes at 90° C. decrosslinking incubation.

### Example 8

Rapid Decrosslinking of FFPE Protein with Catalyst Compounds

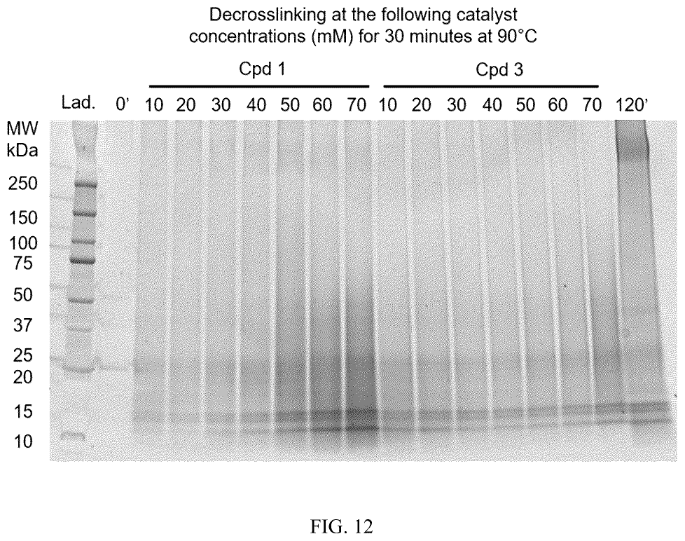

In this example, catalyst effect on FFPE protein yields were next tested. Cpd 1 and Cpd 3 were incubated with FFPE lysate across a range of concentrations (0-70 mM) and pHs (5.3-7.6) and decrosslinked for 30 minutes at 90° C. (Table 3). Soluble protein fractions were analyzed using SDS-PAGE followed by silver staining (FIG. 12). To benchmark protein yields, lysates with no catalyst were extracted alongside, and either not decrosslinked (0′) or decrosslinked for 120 minutes at 90° C. (120′). Cpd 1 and Cpd 3 used at increasing concentrations and lower lysate pHs resulted in greater mobile FFPE protein yields.

TABLE 3
Lysate pH at different concentrations of catalyst

| Catalyst Concentration | Cpd 1 | Cpd 3 |
|---|---|---|
| 10 mM | 7.63 | 7.56 |
| 20 mM | 7.26 | 7.24 |
| 30 mM | 6.70 | 6.86 |
| 40 mM | 6.25 | 6.56 |
| 50 mM | 5.82 | 6.28 |
| 60 mM | 5.56 | 6.04 |
| 70 mM | 5.36 | 5.85 |

### Example 9

### Role of pH for FFPE Protein Decrosslinking

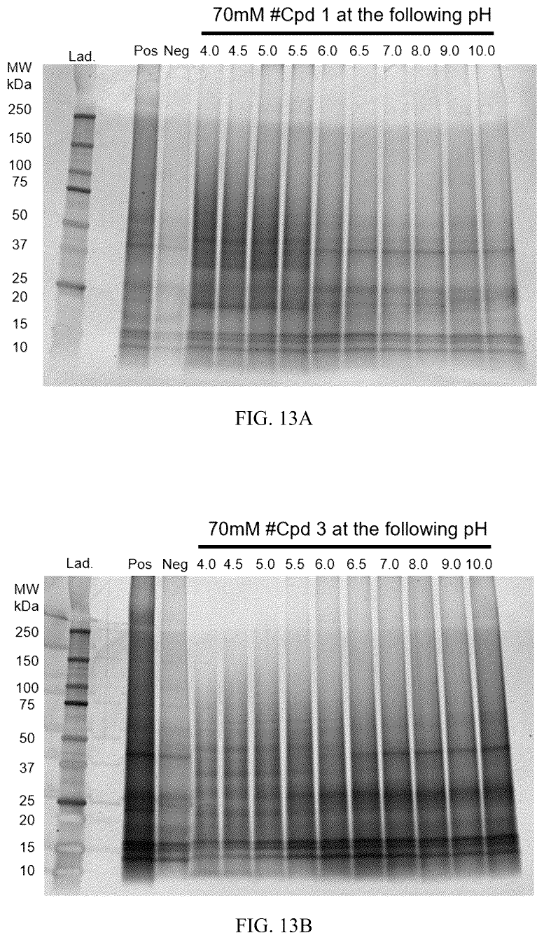

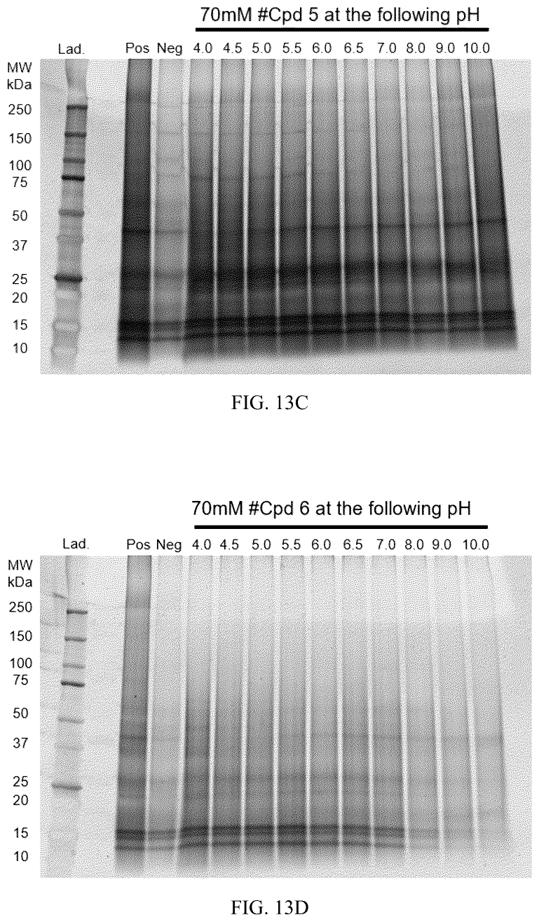

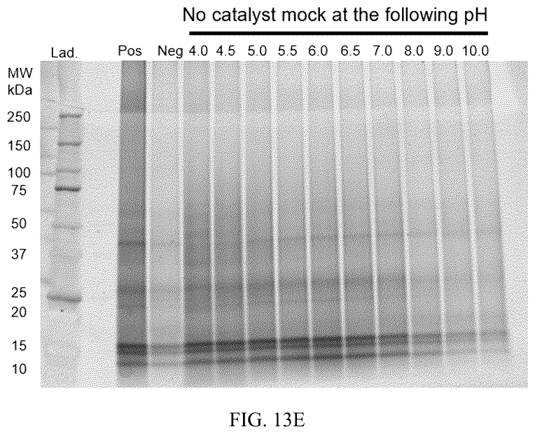

In this example, the role of pH for the rapid FFPE protein decrosslinking reaction was tested. Cpd 1, Cpd 3, Cpd 5, and Cpd 6 were added to lysates at a concentration of 70 mM across a pH range of 4.0 to 10.0, then decrosslinked for 30 minutes at 90° C. To control for the effects of pH on decrosslinking, a no catalyst treatment (using mock additions of H₂O or DMSO) was also added across a pH range of 4.0 to 10.0, then decrosslinked for 30 minutes at 90° C. To benchmark protein yields, lysates with no catalyst were extracted alongside, and either decrosslinked for 120 minutes at 90° C. (Pos) or 30 minutes at 90° C. (Neg). Soluble protein fractions were analyzed using SDS-PAGE followed by silver staining (FIGS. 13A-13E). Lowering the lysate pH was sufficient to increase mobile protein yields, however, the inclusion of the catalysts gave greater yields when compared to the Pos and Neg controls. The catalysts also caused the appearance of higher molecular weight protein bands, which occurred in a pH-dependent manner.

### Example 10

### Role of Catalyst Concentration for FFPE Protein Decrosslinking

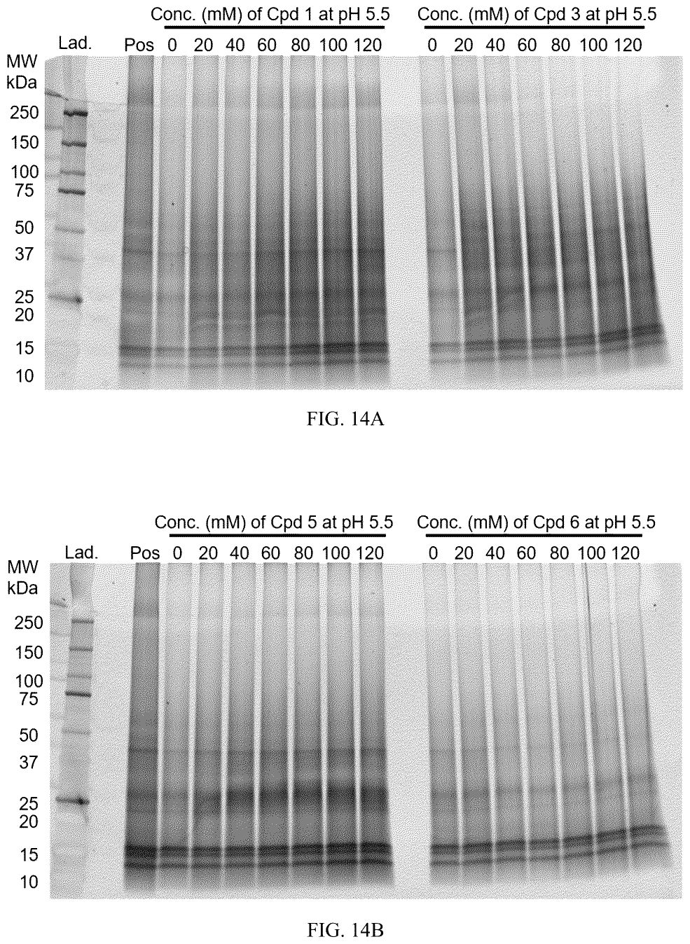

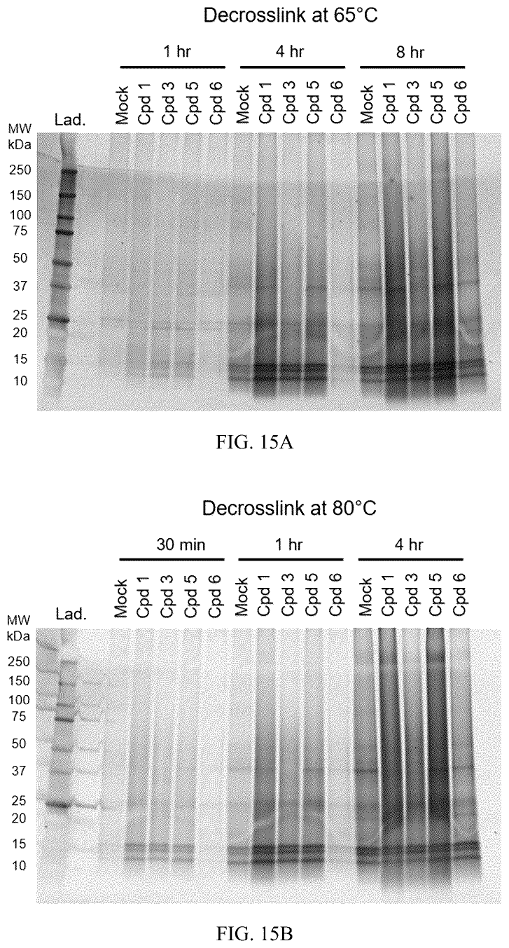

In this example, the role of catalyst concentration for the rapid FFPE protein decrosslinking reaction was tested. Cpd 1, Cpd 3, Cpd 5, and Cpd 6 were added to lysates at concentration of 0 to 120 mM while the pH of the lysate was maintained between 5.4 and 5.6, then decrosslinked for 30 minutes at 90° C. To benchmark protein yields, a lysate with no catalyst decrosslinked for 120 minutes at 90° C. (Pos) was extracted alongside. Soluble protein fractions were analyzed using SDS-PAGE followed by silver staining (FIGS. 14A-14B). Increasing catalyst concentrations led to higher yields of mobile proteins and the appearance of higher molecular weight protein bands.

### Example 11

### Role of Time and Temperature for FFPE Protein Decrosslinking

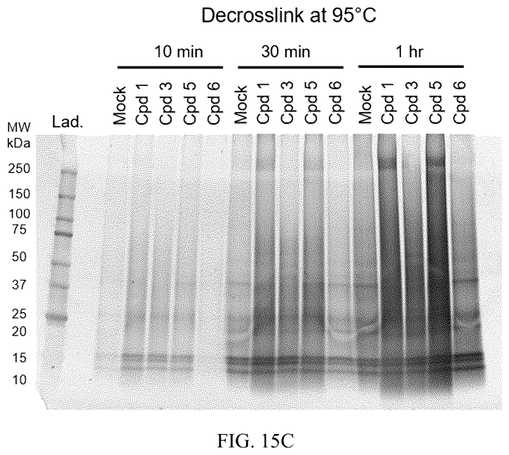

In this example, the time and temperature components of the decrosslinking incubation were next examined. 70 mM of Cpd 1, Cpd 3, Cpd 5, and Cpd 6 were tested at temperatures of 65° C., 80° C., and 95° C., and at three separate time points ranging from 10 minutes to 8 hours. To serve as a control, a no catalyst extraction was performed alongside (mock). Soluble protein fractions were analyzed using SDS-PAGE followed by silver staining (FIGS. 15A-15C). As expected, decrosslinking at higher temperatures and longer incubation times resulted in greater mobile protein yields. The inclusion of catalysts, particularly Cpd 1 and Cpd 5, resulted in higher protein yields compared to the mock at all tested temperatures and time points.

### Example 12

### Catalyst-Decrosslinked FFPE Protein Assessed by Western Blot

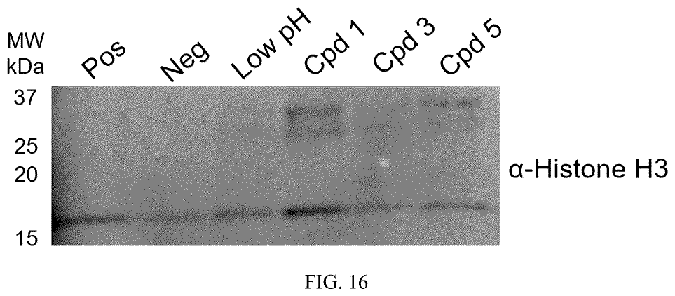

Soluble protein extracts were tested in western blotting using a primary Histone H3 antibody (FIG. 16). A 17 kDa Histone H3 protein band was detected in extracts from Cpd 1, Cpd 3, and Cpd 5. Histone H3 protein bands were also detected in extracts with no catalysts that were decrosslinked for 120 minutes at 90° C. (Pos), 30 minutes at 90° C. (Neg), or 30 minutes at 90° C. with a pH of 5.5 (Low pH).

### Example 13

### LC-MS/MS Analysis of Rapid Decrosslinked FFPE Protein

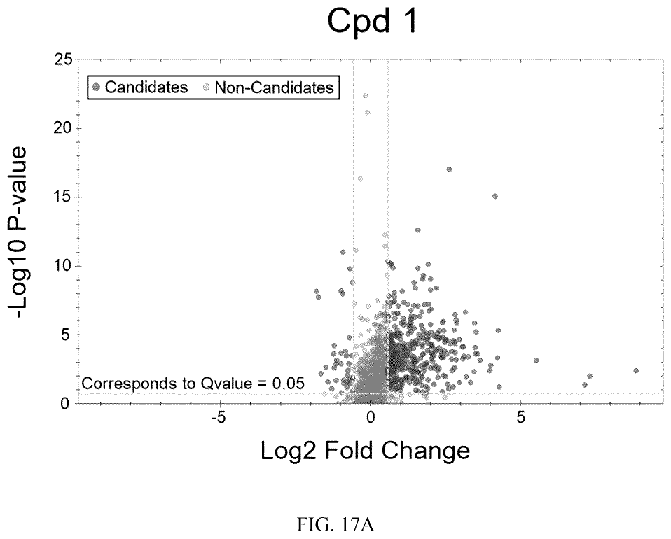

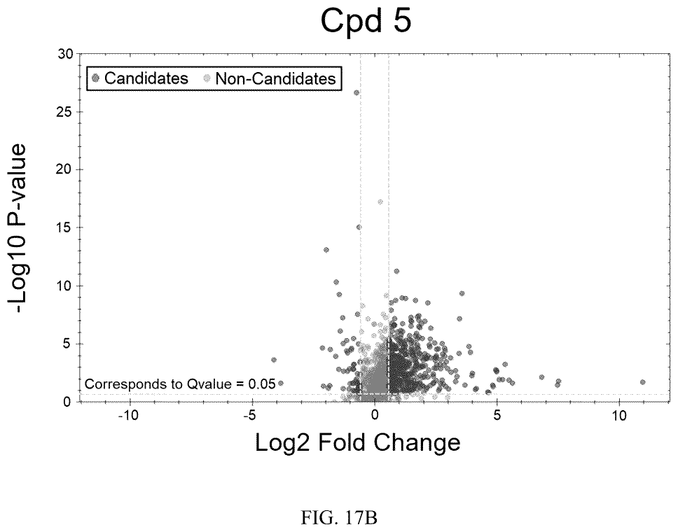

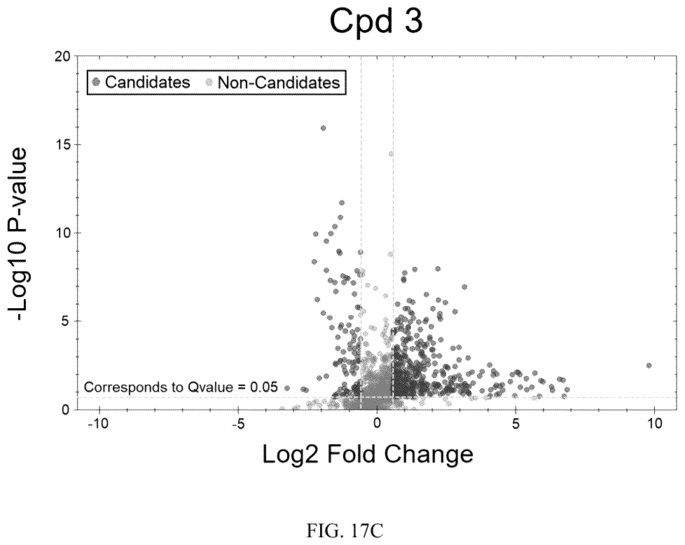

FFPE lysates were rapidly decrosslinked with 70 mM catalyst at pH ˜5.5 (Cpd 1, Cpd 3, Cpd 5) along with a low pH ˜5.5 control, using six extraction replicates for each condition. Total protein extracts were then SP3-purified, desalted, proteolyzed, and subjected to LC-MS/MS as described in Example 9. Protein identity and abundance for each catalyst was plotted over the low pH control and assessed by volcano plots (FIGS. 17A-17C). A significant enrichment of proteins in the catalyst treated samples relative to the control was observed (Log 2 Fold Change, p=0.05).

### Example 14

### Enrichment of Human FFPE Protein Using Rapid Decrosslinking Chemistries

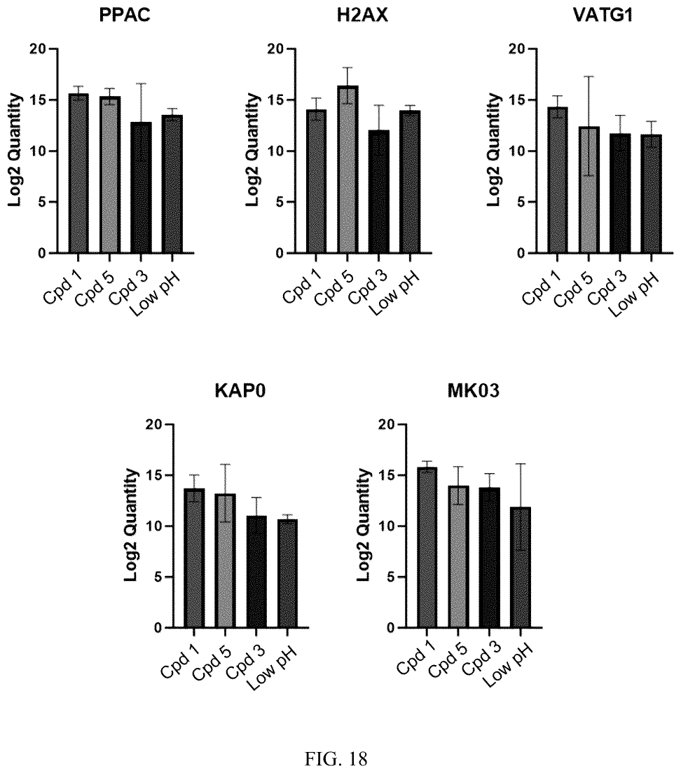

Provided herein are examples of specific human proteins that are more abundantly detected following treatment with catalysts, relative to the low pH control sample (FIG. 18). These values are derived from MS2 chromatographic peak areas (expressed as log 2 Quantity). Significant increases in peak area of proteins are observed with the use of different catalysts.

1. A method of decrosslinking a formaldehyde cross-linked biological sample, comprising contacting the sample with an effective amount of a compound of formula (I): 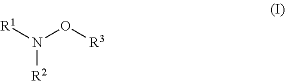
or a salt thereof,
wherein:
(i) R¹ is selected from H, C₁-C₆ alkyl, and carboxy-C₁-C₄-alkyl;
R² is selected from H, C₁-C₆ alkyl, hydroxy-C₁-C₄-alkyl, carboxy-C₁-C₄-alkyl, aryl, heteroaryl, aryl-C₁-C₄-alkyl, and heteroaryl-C₁-C₄-alkyl;
R³ is —X—R⁴, wherein X is selected from —C(O)— and —SO₂—, and R⁴ is selected from C₁-C₆-alkyl, aryl, and heteroaryl;
wherein each aryl and heteroaryl is independently unsubstituted or substituted with 1 or 2 substituents independently selected from halo, hydroxy, C₁-C₄ alkyl, C₁-C₄ alkoxy, amino, amido, carboxy, and ester; and
wherein each alkyl is unsubstituted or substituted with 1 or 2 substituents independently selected from halo, hydroxy, alkoxy, amino, amido, carboxy, ester, optionally substituted C₃-C₆ cycloalkyl, and optionally substituted 3- to 6-membered heterocyclyl;
or
(ii) R¹ is selected from H, C₁-C₆ alkyl, and carboxy-C₁-C₄-alkyl;
R² is selected from C₁-C₆ alkyl, aryl-C₁-C₄-alkyl, heteroaryl-C₁-C₄-alkyl, and —Y—R⁵, wherein Y is selected from a bond, —C(O)—, and —SO₂—, and R⁵ is selected from C₁-C₆ alkyl, aryl, and heteroaryl; and
R³ is H;
wherein each aryl and heteroaryl is independently unsubstituted or substituted with 1 or 2 substituents independently selected from halo, hydroxy, C₁-C₄ alkyl, C₁-C₄ alkoxy, amino, amido, carboxy, and ester; and
wherein each alkyl is unsubstituted or substituted with 1 or 2 substituents independently selected from halo, hydroxy, alkoxy, amino, amido, carboxy, ester, optionally substituted C₃-C₆ cycloalkyl, and optionally substituted 3- to 6-membered heterocyclyl;
or
(iii) R¹ and R², together with the nitrogen atom to which they are attached, are taken together to form an optionally substituted 4- to 8-membered ring; and
R³ is H;
or
(iv) R¹ is H; and
R² and R³, together with the atoms to which they are attached, are taken together to form an optionally substituted 4- to 8-membered ring.

2. The method of claim 1, wherein:
R¹ is H;
R² is selected from C₁-C₆ alkyl, —CH₂-aryl, and —CH₂-heteroaryl; and
R³ is —X—R⁴, wherein X is —C(O)— and R⁴ is selected from C₁-C₆-alkyl, aryl, and heteroaryl;
wherein each alkyl is independently unsubstituted or substituted with 1 substituent selected from hydroxy and carboxy; and
wherein each aryl and heteroaryl is independently unsubstituted or substituted with 1 or 2 substituents independently selected from halo, hydroxy, methoxy, amino, and carboxy.

3. (canceled)

4. The method of claim 1, wherein:
R¹ is H;
R² is selected from aryl-C₁-C₄-alkyl and heteroaryl-C₁-C₄-alkyl, each of which is independently unsubstituted or substituted with 1 or 2 substituents independently selected from halo, hydroxy, methoxy, amino, and carboxy; and
R³ is H.

5. The method of claim 1, wherein:
R¹ and R², together with the nitrogen atom to which they are attached, are taken together form a saturated 4- to 6-membered ring that is unsubstituted or substituted with 1 or 2 substituents independently selected from hydroxy, hydroxy-C₁-C₄ alkyl, C₁-C₄-alkoxy, carboxy, carboxy-C₁-C₄ alkyl, and oxo; and
R³ is H.

6. The method of claim 1, wherein:
R¹ is H; and
R² and R³, together with the atoms to which they are attached, are taken together to form a saturated 4- to 7-membered ring that is unsubstituted or substituted with 1 or 2 substituents independently selected from hydroxy, hydroxy-C₁-C₄ alkyl, C₁-C₄-alkoxy, carboxy, carboxy-C₁-C₄ alkyl, and oxo.

7. The method of claim 1, wherein the compound of formula (I) is selected from: 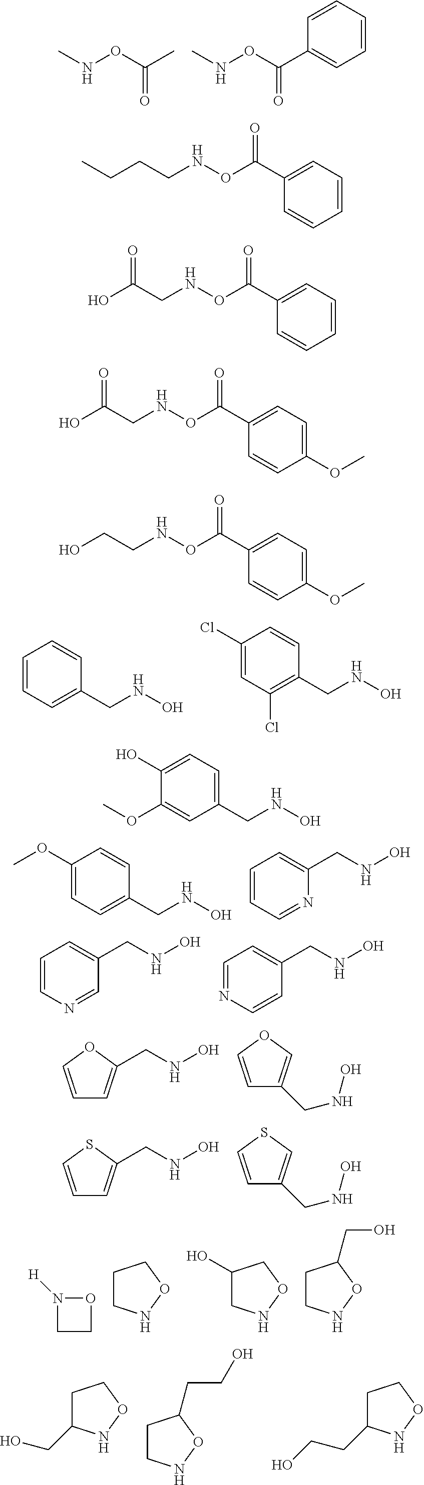 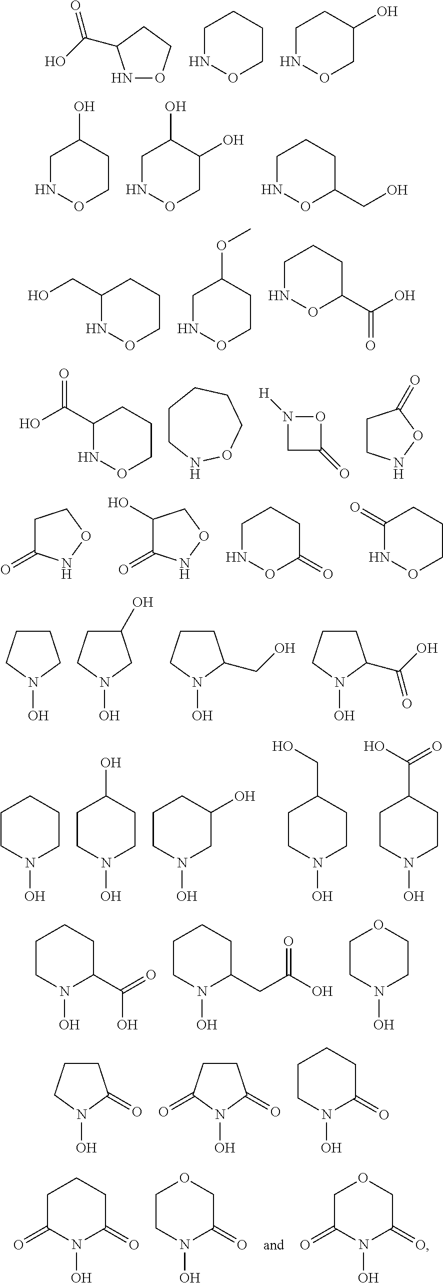
and salts thereof.

8. The method of claim 1, wherein the compound of formula (I) is selected from: 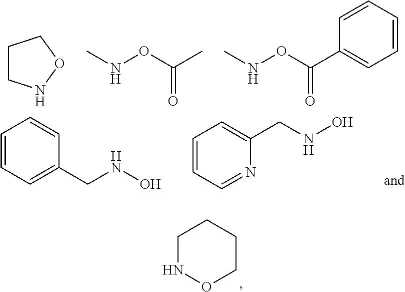
and salts thereof.

9. The method of claim 1, wherein the compound of formula (I) is in the form of a salt.

10. The method of claim 9, wherein the compound of formula (I) is in the form of a hydrochloric acid salt.

11. The method of claim 1, wherein the method comprises contacting the sample with effective amounts of at least two different compounds of formula (I), or salts thereof.

12-13. (canceled)

14. The method of claim 1, wherein the sample is a formalin-fixed paraffin embedded tissue sample, and the method further comprises a step of deparaffinizing the sample prior to contacting the sample with the compound of formula (I), or the salt thereof.

15. (canceled)

16. The method of claim 1, further comprising a step of contacting the sample with a protease prior to contacting the sample with the compound of formula (I), or the salt thereof.

17. The method of claim 16, wherein the protease is proteinase K.

18. The method of claim 1, wherein the contacting step is conducted for about 5 minutes to about 120 minutes and at a temperature of about 20° C. to about 100° C.

19-21. (canceled)

22. The method of claim 1, comprising contacting the sample with a solution of the compound of formula (I), or the salt thereof, and the solution further comprises a buffer selected from tris (hydroxymethyl) aminomethane (Tris), 2-(N-morpholino) ethanesulfonic acid (MES), 4-(2-hydroxyethyl)-1-piperazineethanesulfonic acid (HEPES), phosphate-buffered saline, glycine, and citrate.

23. (canceled)

24. The method of claim 1, comprising contacting the sample with a solution of the compound of formula (I), or the salt thereof, and the resulting solution has a pH of about 4.0 to about 8.5 upon contacting the sample with the solution of the compound of formula (I).

25. (canceled)

26. The method of claim 1, comprising contacting the sample with a solution of the compound of formula (I), or the salt thereof, wherein the solution comprises the compound of formula (I) at a concentration of about 1 mM to about 120 mM.

27. (canceled)

28. The method of claim 1, further comprising extracting one or more components from the sample after the contacting step, wherein the one or more components are selected from nucleic acids and proteins.

29-33. (canceled)

34. A composition comprising:
a formaldehyde cross-linked biological sample; and
a compound of formula (I): 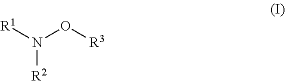
or a salt thereof,
wherein:
(i) R¹ is selected from H, C₁-C₆ alkyl, and carboxy-C₁-C₄-alkyl;
R² is selected from H, C₁-C₆ alkyl, hydroxy-C₁-C₄-alkyl, carboxy-C₁-C₄-alkyl, aryl, heteroaryl, aryl-C₁-C₄-alkyl, and heteroaryl-C₁-C₄-alkyl;
R³ is —X—R⁴, wherein X is selected from —C(O)— and —SO₂—, and R⁴ is selected from C₁-C₆-alkyl, aryl, and heteroaryl;
wherein each aryl and heteroaryl is independently unsubstituted or substituted with 1 or 2 substituents independently selected from halo, hydroxy, C₁-C₄ alkyl, C₁-C₄ alkoxy, amino, amido, carboxy, and ester; and
wherein each alkyl is unsubstituted or substituted with 1 or 2 substituents independently selected from halo, hydroxy, alkoxy, amino, amido, carboxy, ester, optionally substituted C₃-C₆ cycloalkyl, and optionally substituted 3- to 6-membered heterocyclyl;
or
(ii) R¹ is selected from H, C₁-C₆ alkyl, and carboxy-C₁-C₄-alkyl;
R² is selected from C₁-C₆ alkyl, aryl-C₁-C₄-alkyl, heteroaryl-C₁-C₄-alkyl, and —Y—R⁵, wherein Y is selected from a bond, —C(O)—, and —SO₂—, and R⁵ is selected from C₁-C₆ alkyl, aryl, and heteroaryl; and
R³ is H;
wherein each aryl and heteroaryl is independently unsubstituted or substituted with 1 or 2 substituents independently selected from halo, hydroxy, C₁-C₄ alkyl, C₁-C₄ alkoxy, amino, amido, carboxy, and ester; and
wherein each alkyl is unsubstituted or substituted with 1 or 2 substituents independently selected from halo, hydroxy, alkoxy, amino, amido, carboxy, ester, optionally substituted C₃-C₆ cycloalkyl, and optionally substituted 3- to 6-membered heterocyclyl;
or
(iii) R¹ and R², together with the nitrogen atom to which they are attached, are taken together to form an optionally substituted 4- to 8-membered ring; and
R³ is H;
or
(iv) R¹ is H; and
R² and R³, together with the atoms to which they are attached, are taken together to form an optionally substituted 4- to 8-membered ring.

35-47. (canceled)

48. A kit comprising:
(A) a compound of formula (I): 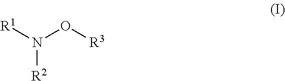
or a salt thereof,
wherein:
(i) R¹ is selected from H, C₁-C₆ alkyl, and carboxy-C₁-C₄-alkyl;
R² is selected from H, C₁-C₆ alkyl, hydroxy-C₁-C₄-alkyl, carboxy-C₁-C₄-alkyl, aryl, heteroaryl, aryl-C₁-C₄-alkyl, and heteroaryl-C₁-C₄-alkyl;
R³ is —X—R⁴, wherein X is selected from —C(O)— and —SO₂—, and R⁴ is selected from C₁-C₆-alkyl, aryl, and heteroaryl;
wherein each aryl and heteroaryl is independently unsubstituted or substituted with 1 or 2 substituents independently selected from halo, hydroxy, C₁-C₄ alkyl, C₁-C₄ alkoxy, amino, amido, carboxy, and ester; and
wherein each alkyl is unsubstituted or substituted with 1 or 2 substituents independently selected from halo, hydroxy, alkoxy, amino, amido, carboxy, ester, optionally substituted C₃-C₆ cycloalkyl, and optionally substituted 3- to 6-membered heterocyclyl;
or
(ii) R¹ is selected from H, C₁-C₆ alkyl, and carboxy-C₁-C₄-alkyl;
R² is selected from C₁-C₆ alkyl, aryl-C₁-C₄-alkyl, heteroaryl-C₁-C₄-alkyl, and —Y—R⁵, wherein Y is selected from a bond, —C(O)—, and —SO₂—, and R⁵ is selected from C₁-C₆ alkyl, aryl, and heteroaryl; and
R³ is H;
wherein each aryl and heteroaryl is independently unsubstituted or substituted with 1 or 2 substituents independently selected from halo, hydroxy, C₁-C₄ alkyl, C₁-C₄ alkoxy, amino, amido, carboxy, and ester; and
wherein each alkyl is unsubstituted or substituted with 1 or 2 substituents independently selected from halo, hydroxy, alkoxy, amino, amido, carboxy, ester, optionally substituted C₃-C₆ cycloalkyl, and optionally substituted 3- to 6-membered heterocyclyl;
or
(iii) R¹ and R², together with the nitrogen atom to which they are attached, are taken together to form an optionally substituted 4- to 8-membered ring; and
R³ is H;
or
(iv) R¹ is H; and
R² and R³, together with the atoms to which they are attached, are taken together to form an optionally substituted 4- to 8-membered ring; and
(B) instructions for decrosslinking a formaldehyde cross-linked biological sample by contacting the sample with the compound of formula (I), or the salt thereof.

49-62. (canceled)
